(19) United States
(12) Patent Application Publication
(10) Pub. No.: US 2004/0235725 A1
(43) Pub. Date: Nov. 25, 2004

(54) Methods for making pectin-based mixed polymers

(76) Inventors: Peter Albersheim, Athens, GA (US); Ivana Djelineo-Albersheim, Athens, GA (US); Alan Darvill, Bogart, GA (US)

Correspondence Address: Greenlee Winner and Sullivan, 5370 Manhattan Circle, Suite 201, Boulder, CO, 80303, US

(21) Appl. No.: 10/487,753

(22) PCT Filed: Sep. 3, 2002
(86) PCT No.: PCT/US02/28066

Related U.S. Application Data
(60) Provisional application No. 60/316,777, filed on Aug. 31, 2001.

(51) Int. Cl. C12P 21/06; A61K 38/00

(52) U.S. Cl. 514/12; 435/68.1

(57) ABSTRACT

This application discloses that two enzymes formerly known in the art as pectin methylesterase (PME) and endopolygalacturonidase (EPG) possess additional catalytic activities as pectin transester synthase (PTES) and pectin transesterase (PTE), respectively. The PTES catalizes the synthetic reaction that covalently cross-links homogalacturonan chains in the primary cell wall via ester bonds. Thus PTES can be employed to form at least one ester bond between two chemical entities, one carrying at least one acid, salt of an acid, or ester group, and one carrying at least one hydroxyl group, e.g., between two polymers, a polymer and a monomeric compound or two monomeric compounds. Further PTES can be employed to form at least one amide bond between two chemical entities, one carrying at least one acid, or ester group, and one carrying at least one amine group, preferably an unsubstituted amine group (—N₂). The pectin transesterase (PTE) disclosed herein catalyzes the hydrolysis or ester bonds between the carboxyl group(s) of galactosyluronic acid residues of one homogalacturonan) chain and the O-2 and/or O-3 hydroxyl group(s) of galacturonic acid residues of another homogalacturonan. PTE activity is shown to reduce the viscosity of pectin solutions in vitro. Thus, the PTE enzyme can be used as an additive to modify the fluidity of a variety of food and pharmaceutical preparations containing pectin, in particular, juice, pastes, jellies, and jams.

### BACKGROUND OF THE INVENTION

The present invention relates to methods of producing pectin-based mixed polymers and regulating the viscosity of pectin-containing gels using newly identified enzymatic activities derived from plants.

The primary cell walls of all higher plants, i.e., the walls of the succulent tissues, are composed of six structurally defined polysaccharides i.e., cellulose, xyloglucan, arabinoxylan, homogalacturonan (HG), rhamnogalacturonan-l (RG-l), and, unrelated, rhamnogalacturonan-ll (RG-ll). Some primary cell walls of cereals also contain 3- and 4-linked β-glucan. Many primary cell walls also contain structural protein. Even though the primary cell wall polysaccharides are an important component of man's diet, constituting the principal component of dietary fiber, they have not been widely studied. Although the general features of the primary structures of the six polysaccharides are well established, no polysaccharide present in the cell walls of plants has ever been synthesized in a cell-free system. Indeed, even the mechanisms by which plant cell wall polysaccharides are biosynthesized have not been elucidated, although it is widely accepted that cellulose is synthesized by enzymes located in the plasma membrane and that the other five primary cell wall polysaccharides are synthesized by enzymes located in the Golgi.

Structural characterization of the primary cell wall polysaccharides has provided much insight into their primary structures and some information about how the polysaccharides interact within the cell wall. Xyloglucan and arabinoxylan, the two hemicelluloses of primary cell walls, have cellulose-like backbones that enable them to hydrogen bond strongly to and presumably cross-link cellulose microfibrils. Strong alkali is required to solubilize even a fraction of the xyloglucan that binds in a test tube to pure cellulose.

The cellulose-xyloglucan (and presumably arabinoxylan) complex is viewed as one of two matrices that constitute the principal structural components of all primary cell walls. The second matrix is composed of cross-linked pectic polysaccharides. It is believed that these two matrices form a largely co-extensive gel.

Pectic polysaccharides containing extensive stretches of unesterified galactosyluronic acid resides, are believed to be important components of the middle lamella, which is the area between cells where the walls of neighboring cells come together. The middle lamella is a barrier to movement of polysacharides and, presumably, of many other molecules from one cell wall to its neighbor.

The pectin gel includes the three pectic polysaccharides, homogalacturonan (HG), RG-I and RG-II, interconnected by glycosidic and ester bonds. Treatment of primary cell walls with a pure endopolygalacturonase (EPG), which hydrolyses only the glycosidic linkages of unesterified galactosyluronic acid residues of homogalacturonan (HG), releases RG-I and RG-II as well as homogalacturonan fragments. This result indicates that these polysaccharides are interconnected through endopolygalacturonase-susceptible glycosidic bonds, leading us to conclude that the three pectic polysaccharides are interconnected.

The pectic gels of primary cell walls appear to utilize intermolecular cross-links, perhaps in part through the formation of Ca²⁺ coordination bonds. Pectins indeed form gels, and the mechanism of gelation has been the subject of numerous studies [MacDougall et al. (1996) Carbohydr. Res. 293:235-249]. In general, two classes of gelation are achieved with pectin. The type of gelation depends on the pectic methyl ester content and is exemplified by high methoxy and low methoxy pectin gels.

Solutions of high methoxy pectins, which have 50% or more of their carboxyl groups methylesterified, can be induced to gel at pH values at or below 3.5 by the addition of large quantities (˜50% by weight) of a low molecular weight carbohydrate, such as sucrose. Gels formed this way are called pectin-sugar-acid gels [Pilnik et al. (1992) Advances in Plant Cell Biochemistry and Biotechnology 1:219-270]. The high content of low molecular weight carbohydrate reduces the activity of the water, which promotes chain-chain interactions rather than chain-solvent interactions [Rees, D. A. (1972) Chem, Industry 630-636]. It has been hypothesized that gelling is initiated through the formation of junction zones consisting of three to ten co-operatively-ordered chains linked together in the form of a three-dimensional network [Walkshaw et al. (1981) J. Mol. Biol. 153:1075-1085]. Water molecules that surround the methyl groups are disrupted by the high content of low molecular weight carbohydrate, thus forcing the methyl groups to turn to hydrophobic environments. At the low pH the pectic carboxyl groups are protonated, which in effect “lowers the coulombic repulsion between chains” and stabilizes junction zones [Oakenfull et al. (1984) J. Food Sci. 49:1093-1098]. Thus both hydrophobic interactions and coulombic repulsions influence the strength of the three dimensional network formed by the process of pectin gelation.

Different low molecular weight carbohydrates, such as glucose and fructose, and other compounds like ethanol, t-butanol and dioxane [Oakenfull et al. (1984) supra], even at concentrations that provide the same low water activity, vary in their ability to promote the formation of high methoxy pectin gels. This variability has been explained by the dependence of hydrogen bonds on the molecular weight of the carbohydrate or compound in question, by the compound's influence on the water activity of the system, and by the compound's hydrophobicity effects, which depend on the spacing and orientation of the compound's hydroxyl groups [Pilnik et al. (1992) supra].

Low methoxy pectin or homogalacturonan (HG), with less than 50% of its carboxyl groups methylesterified, can be induced to gel in the presence of 30-60 mg of Ca²⁺ per gram of pectin [Pilnik et al. (1992) supra]. Calcium pectate gels are thermoreversible. Calcium can be added to an 80° C. solution of pectin that will gel upon cooling [Powell, et al. (1982) J. Mol. Biol. 155:517-531]. Calcium-promoted gelation occurs in HG chains that contain blocks of contiguous, unesterified galactosyluronic acid residues [Tuerena et al. (1982) Carbohydr. Polym. 2:193-203]. In the case of pectins with a random distribution of unesterified galactosyluronic acid residues [Thibault et al. (1986) Biopolymers 25:455-468], the percent of carboxyl groups methylesterified must fall below 40% before calcium-ion induced gelation occurs.

Calcium pectate gels are thought to form in a similar manner to low water activity pectin-sugar-acid gels. A network is thought to form from HG molecules in which the solvent is suspended. Calcium is thought to cross-link HGs that have a stretch of 14 unesterified galactosyluronic acid residues [Powell et al. (1982) supra]. A recent molecular dynamics study [Manunza et al. (1998) Glycoconj J. 15:297-300] of the interactions of calcium and sodium ions with polygalacturonate chains indicated that the formation of calcium bridges between polygalacturonate chains is possible. A calcium-polygalacturonate complex was calculated to have lower energy than a sodium-polygalacturonate complex, indicating that the calcium complex is thermodynamically preferable. However, these molecular dynamic simulations were based on linear, completely de-esterified oligogalacturonides with just 12 galactosyluronic acid residues. Thus, it remains to be determined whether these studies reflect the situation in living tissues, as HG chains in plant cell walls are thought to be much longer than 12 residues and are quite heavily methylesterified. Calcium has to be added slowly to low methoxy pectin in order to form a calcium-pectate gel. A calcium pectate gel can also be formed by slow pectin methyl-esterase (PME) de-esterification of a high methoxy pectin. Gels formed in response to PME are a phenomenon known in the citrus industry, where concentrated fruit juices form an undesirable gel as a result of the action of residual endogenous PME [Pilnik et al. (1992) supra].

The in vitro gelation of pectins isolated from peach fruit [Zhou et al. (2000) Phytochemistry 55:191-195] has been investigated. In particular, studies have been conducted on the effect on gelling of adding various amounts of endopolygalacturonase (EPG) and PME to solutions of water, trans-1,2-diaminocyclohexane-N,N,N′,N′-tetraacetic acid- (CDTA), and carbonate-extractable pectins. Only the CDTA-soluble fraction formed a gel in two days in the presence of PME. However, the authors have not given an explanation for the mechanism by which CDTA-soluble pectin gels in the presence of PME. The consensus of those working in the pectin field is that gelation occurs because of the presence of calcium ions in plant extracts. This is hard to reconcile with the fact that it is the CDTA-extractable pectin, in particular, that can be induced to gel. CDTA is an excellent chelator of calcium. Furthermore, CDTA and calcium would likely have been at least partially removed from the gelling solution by dialysis at 4° C. against six changes of double-distilled water.

Recent studies have suggested the presence of esters, other than methyl esters, in various pectin preparations. Kim and Carpita (1992) [Plant Physiol. 98:646-653] reported that approximately 65% of the galactosyluronic acid residues in the walls of unextended coleoptiles are esterified and only about two-thirds of those esters are accounted for by methyl esters. The proportion of galactosyluronic acid residues that are methyl esterified decreased throughout elongation of the coleoptile. Thus, methyl esters could not account for the observed increase from 65% to 80% of the galactosyluronic acid carboxyl groups that are esterified as the coleoptiles complete elongation. McCann et al. (1994) [Plant J. 5:773-785] also reported that methyl esters account for only a portion of the total esterified galactosyluronic acid residues in the cell walls of both unadapted and sodium chloride-adapted tobacco cells in suspension culture. In their studies, the total ester content of unadapted tobacco cells in suspension culture increased from about 50% in dividing cells to 78% during elongation. However, methyl esters accounted for only half of the total esters during the cell division phase and about two-thirds during elongation. Doong et al. (1996) [Plant Physiol. 109:141-152] further showed that methyl esters cleaved by A. niger PME accounted for only about 40% of the esters present in HG.

The present application discloses two newly identified enzymatic activities called pectin transesterase (PTE) and pectin transesterase synthase (PTES) that are responsible for hydrolyzing and synthesizing such ester bonds between HG chains of pectin and the uses thereof

### SUMMARY OF THE INVENTION

This invention is based on the inventors' discovery that two enzymes formerly known in the art as pectin methylesterase (PME) and endopolygalacturonidase (EPG) possess additional catalytic activities as pectin transester synthase (PTES) and pectin transesterase (PTE), respectively.

The PTES catalizes the synthetic reaction that covalently cross-links homogalacturonan chains in the primary cell wall via ester bonds. Thus PTES can be employed to form at least one ester bond between two chemical entities, one carrying at least one acid, salt of an acid, or ester group, and one carrying at least one hydroxyl group, e.g., between two polymers, a polymer and a monomeric compound or two monomeric compounds. Further PTES can be employed to form at least one amide bond between two chemical entities, one carrying at least one acid, salt of an acid, or ester group, and one carrying at least one amine group, preferably an unsubstituted amine group (—NH₂). In a preferred embodiment, the PTES can be employed to form at least one ester or amide bond between two polymers or between a polymer and a monomeric compound. The formation of one or more ester or amide bonds between two polymers can be employed to generate cross-linked polymers, affecting, for example, the rheological properties of the cross-linked material. The formation of one or more ester or amide bonds between a polymer and a monomeric compound can be employed, for example, to generate a derivatized polymer having selected desirable properties conferred by derivitization with the monomeric compound.

Specifically, the PTES of the present invention provides a new method of producing pectin-based mixed polymers by cross-linking homogalacturonan carrying acid groups with polymer molecules or monomeric molecules carrying hydroxyl or amine groups via the formation of intermolecular ester or amide bonds. Examples of monomeric compounds and polymers carrying hydroxyl or amide groups that can be used in the invention include, but are not limited to any unsubstituted amines, alcohols, putrescene, spermine, spermidine, proteins, sugars, polysaccharides, carbohydrate, hydroxylated long-chain fatty acids, inositol-containing compounds, phosphoinositol membrane anchors, nucleic acids (e.g. RNA) or derivatives thereof. The PTES is also useful in making a pectic gel consisting of homogalacturonans or of a mixture of homogalacturonan and any other polysaccharides in the absence of calcium via cross-linking.

The pectic gel made according to the invention containing little or very little levels of calcium can be used as a gelling agent in foodstuffs, pharmaceuticals, and nutritional products.

Pectin transester synthase can be from any source that shows PME activity, preferably a plant, and purified by the art-known methods used for purifying PME from plants such as tomato, tobacco and spinach. Alternatively, the PTES can be made by any art known recombinant methods.

The pectin transesterase (PTE) disclosed herein catalyzes the hydrolysis of ester bonds between the carboxyl group(s) of galactosyluronic acid residues of one homogalacturonan chain and the O-2 and/or O-3 hydroxyl group(s) of galactosyluronic acid residues of another homogalacturonan. PTE activity is shown to reduce the viscosity of pectin solutions in vitro. Thus, the PTE enzyme can be used as an additive to modify the fluidity of a variety of food and pharmaceutical preparations containing pectin, in particular, juice, pastes, jellies, and jams. The PTE enzyme is also useful for removing undesired polymers or gels containing ester bonds, e.g., as an additive in a cleaning solution. One outcome of such PTE activity in plant cell walls is softening of the fruit for ripening. Therefore, this invention provides a new means of regulating the ripening process by modulating PTE activity. The PTE enzyme can be isolated from any plant source by employing the protocol disclosed in the present application.

### BRIEF DESCRIPTION OF THE DRAWINGS

FIG. 1 shows the ester cross-links in homogalacturonan (HG) chains. Ester cross-links can be formed between the carboxyl groups of galacturonic acid residue(s) in one HG chain and the O-2 and/or O-3 hydroxyl groups of galacturonic acid residue(s) in another HG chain.

FIG. 2 illustrates the profile of gel-permeation chromatography (Bio-Gel P-30) of endopolygalacturonidase (EPG)-solubilized cell wall components of suspension-cultured sycamore cells. Column fractions were assayed for neutral residues by the anthrone method (A₆₂₀=∘), for uronic acid residues by the metahydroxybiphenyl method (A₅₂₀=●), and for KDO residues by the modified thiobarbituric acid method (A₅₄₈=⋄). Column fractions 13 to 30 were combined as fraction A, and column fractions 31-51 were combined as fraction B (Marfa et al. (1991) Plant J. 1:219).

FIG. 3 shows the profile of gel-permeation chromatography of fraction A from the Bio-Gel P-30 column in FIG. 2. The sample contained in fraction A was deesterified and rechromatographed on the Bio-Gel P-30. Column fractions were assayed as in FIG. 2. Column fractions 14 to 18 were combined as fraction A1, fractions 19 to 25 as fraction A2, fractions 26 to 41 as fraction A3, and fractions 42-54 as fraction A4 (Marfa et al. (1991) Plant J. 1:219).

FIG. 4 shows the degree of polymerization of oligogalacturonidases contained in fraction A3 and A4 of FIG. 3 as measured by Dionex HPAE-PAD (Marfa et al. (1991) Plant J. 1: 219).

FIG. 5 shows the PAGE analysis of cold base-treated fraction A stained with alcian blue and silver. Lane 1 is the sample without the cold base treatment. Lane 2 shows a mixture of oligogalacturonides with DPs from approximately 6 to 20. Those oligogalacturonides with DPs less than 10 have migrated off the gel. Lane 3 shows the sample treated with cold base. Note that there is more mRG-II and dRG-II than lane 1, indicating that the cold base treatment hydrolyzed covalent links between RG-II and HG.

FIG. 6 shows the results of the PAGE assay of EPG and cold base-treated commercial pectins stained with alcian blue and silver. Lane 1: untreated Sigma pectin, 67% methyl-esterified; lane 2: EPG-treated Sigma pectin; lane 3: Sigma pectin treated with EPG and cold base; lane 4: cold base-treated Sigma pectin; lane 5: untreated Hercules pectin, 73% methyl-esterified; lane 6: EPG-treated Hercules pectin; lane 7: Hercules pectin treated with EPG and cold base; lane 8: Hercules pectin treated with cold base.

FIG. 7 shows the results of the PAGE analysis of Hercules pectin treated with cold base for various lengths of time. Hercules pectin was treated with cold base at 4° C., pH 12 for: untreated (lane 1), 30 minutes (lane 2), 1 hour (lane 3), 2 hour (lane 4), 4 hour (lane 5). Lanes 6 and 7 represent samples treated at 4° C., pH 12 for 4 hour and then room temperature for 90 minutes and 3 h, respectively.

FIG. 8 shows the absorbance profile at 235 nm of Hercules pectin treated with cold base for various lengths of time. Hercules pectin (73% methyl esterified) was treated with cold base at 4° C., pH 12 for: untreated (lane 1), 30 minutes (lane 2), 1 hour (lane 3), 2 hour (lane 4), 4 hour (lane 5). Lanes 6 and 7 represent samples treated at 4° C., pH 12 for 4 hour and then room temperature for 90 min, respectively.

FIG. 9 shows the viscosity measurement of Hercules pectin treated with cold base for various lengths of time. Hercules pectin (73% methyl esterified) was treated with cold base at 4° C., pH 12 for: untreated (lane 1), 30 minutes (lane 2), 1 hour (lane 3), 2 hour (lane 4), 4 hour (lane 5). Lanes 6 and 7 represent samples treated at 4° C., pH 12 for 4 hour and then room temperature for 90 min, respectively.

FIG. 10 shows the results of the pectin transesterase (PTE) assay in various fruit extracts. Extracts of plum, peach, and tomato prepared as shown in Scheme 1 were assayed for their ability to release oligogalacturonide fragments from fraction A. Forty μl of each extract was lyophilized and dissolved in 8 μl of water. Two μl of fraction A (1 mg/ml) was added to each fruit extract. The resulting mixture was allowed to react for 2 hour at 37° C. (lanes labeled A) or at room temperature (lanes labeled C). The reactions in which the fruit extract was boiled for 5 minutes before mixing with fraction A are shown in lanes labeled B.

FIG. 11 shows that pectin methylesterase (PME) does not release oligogalacturonides from Hercules pectin whereas the combination of PME and PTE does. Hercules pectin (5 μl of 1 mg/ml in 50 mM sodium acetate, pH 5.2) was mixed with: 5 μl of 50 mM sodium acetate buffer as control (lane 1): 0.5 μl of GT-PME and 4.5 μl of 50 mM sodium acetate buffer (lane 2), 0.5 μl of GT-PME and 4.5 μl of purified PTE (lane 3). The reaction mixtures were incubated for 3 hour at room temperature before analysis.

FIGS. 12A and 12B show that pectin transesterase (PTE) does not bind to an anion-exchange column whereas an enzyme with EPG-like activity does bind to the column. FIG. 12A shows the PAGE analysis of the HiTrap Q flow-through (QFT). The QFT fraction was assayed by mixing 5 μl of Hercules pectin (1 mg/ml) with the indicated volume of the fraction in 50 mM sodium acetate, pH 5.2. FIG. 12B shows the PAGE analysis of the Q bound (QB) fraction. The QB fraction was dialyzed against 50 mM sodium acetate, pH 5.2, and each aliquot was adjusted to the volume of the original QB fraction by diluting 3.9 fold. The assay conditions are as described in the Examples Section.

FIG. 13 shows the elution profile of the HiTrap Heparin column of the protein extracts prepared from tomatoes. The QFT fraction was applied to a HiTrap heparin column and the proteins were eluted with a linear gradient of 0-0.35 M NaCl in 50 mM sodium acetate, pH 5.2. The PTE activity was eluted in fractions 35 to 45.

FIGS. 14A-14C show the results of the assays of the HiTrap heparin column fractions 34 to 50 (see FIG. 11) for PTE, PME, and EPG. FIG. 14A shows the assay results of every second column fraction from 34 to 50 for PTE, FIG. 14B shows the results of the same fractions for PME, and FIG. 14C shows the results of the PAGE analysis of the same fractions. The arrow in FIG. 14C indicates a band of approximately 45 kDa that correlates well with the fractions containing the PTE activity.

FIG. 15 shows the elution profile from Superdex 75 column. The fractions containing PTE activity from the HiTrap heparin column (see FIGS. 13 and 14A) were applied to a Superdex 75 size-exclusion chromatography column. Proteins were eluted with 0.35 M sodium chloride in 50 mM sodium acetate, pH 5.2. The eluant was monitored by the absorption of ultraviolet light at 280 nm.

FIG. 16 shows the results of the PTE assay of the fractions (12-36) that eluted from the Superdex 75 column. Five μl aliquots of Hercules pectin (1 mg/ml) was combined with 0.5 μl of GT-PME, 4 μl of 50 mM NaAc, pH 5.2, and 0.5 μl of a fraction as indicated and the PTE activity was measured as described herein.

FIG. 17 shows the PAGE (4-15%) analysis of fraction 22 from the Superdex 75 column.

FIG. 18 shows the amino acid sequence of endopolygalacturonase isolated from a ripe tomato. This sequence is taken from the Genbank database (Accession No. 1403396A). The highlighted sequences represent those of the eight peptides derived from the purified PTE that were used to search the database.

FIG. 19 illustrates the ability of the purified PTE of the invention and the fugal EPG to degrade a mixture of unesterified oligogalacturonides that have various degrees of polymerization from 8 to 20. Lanes 1 and 2 are controls (no enzyme added); lanes 3-7 are samples with 0.5 μl of tomato PTE; lanes 8-12 are samples with 0.5 μl of fungal EPG.

FIGS. 20A and 20B show the comparison between tomato PTE and tomato EPG for their ability to degrade an OGmix containing oligogalactosyluronic acid with 8 through 20 galacturonic acid residues (FIG. 20A), and a preparation containing 14 galacturonic acid residues (14-mer) (FIG. 20B). Five μl of OGmix (1 mg/ml) was mixed with: 5 μl of 50 mM sodium acetate, pH 5.2, buffer only (lanes 1 and 4); 0.5 μl of PTE plus 4.5 μl buffer (lanes 2 and 5); 0.5 μl of tomato EPG (diluted 8 times) plus 4.5 μl buffer (lanes 3 and 6); Hercules pectin with 5 μl of buffer only (lanes 7 and 9); Hercules pectin with 0.5 μl of PTE, 0.5 μl of GT-PME plus 4 μl buffer (lanes 8 and 10). FIG. 20B shows the results with the 14-mer: 2 μl of 14-mer (1 mg/ml) with the buffer only (lanes 1 and 4); 2 μl of 14-mer with 0.5 μl of PTE (lanes 2 and 5); 2 μl of 14-mer with 0.5 μl of tomato EPG (diluted 8 times) (lanes 3 and 6).

FIG. 21 is a scheme illustrating the synthesis of ester cross-links as catalyzed by pectin transester synthase (PTES).

FIGS. 22A and 22B show examples of a pectin gel formed by the action of PME/PTES using 10% methyl-esterified Sigma pectin (FIG. 22A) and 73% methyl-esterified Hercules pectin (FIG. 22B).

FIGS. 23A and 23B show the elution profile from a hydrophobic interaction column. FIG. 23A is the protein profile eluted from Phenyl Superose column with 25 ml of decreasing gradient of 1.7 to 0 M ammonium sulfate in 50 mM sodium acetate, pH 5.2. The column fractions containing PME activity are indicated with a dashed line. FIG. 23B shows the protein profile of the Phenyl Superose column fractions (every second fractions as shown on top).

FIGS. 24A and 24B show the elution profile of tomato extracts from Superdex 75 column. FIG. 24A shows the peak eluted with 50 mM Tris, pH 7.5, containing 0.5M NaCl. FIG. 24B shows the protein profile of column fractions 20 through 25.

### DETAILED DESCRIPTION OF THE INVENTION

In general, the terms and phrases used herein have their art-recognized meaning, which can be found by reference to standard textbooks, journal references, and contexts known to those skilled in the art.

The term monomeric compound is used generally herein to encompass any non-polymeric material which does not contain repeated monomer units. A monomeric compound can be a monomer, such as a monosaccharide including those containing acid or amine groups, e.g., a sugar acid such as galacturonic acid, an amino acid, an aliphatic or aromatic alcohol, an aliphatic or aromatic primary or secondary amine, an aliphatic or aromatic ester, an aliphatic or aromatic acid, or salt thereof.

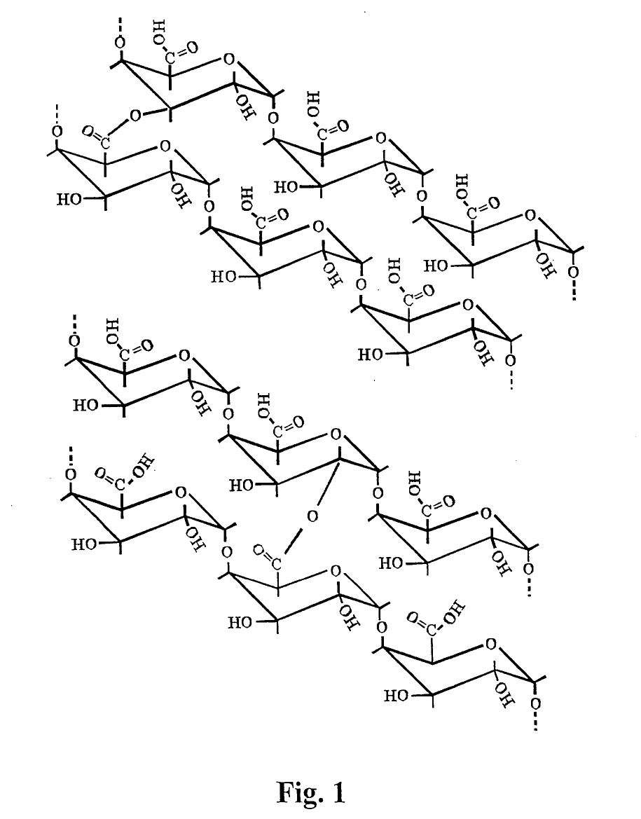

The inventors of the present application initiated studies to establish the existence of ester bonds other than methyl esters in galactosyluronic acid residues in pectin and to identify the enzymes responsible for creating and degrading such ester bonds. As shown in FIG. 1, ester cross-links can be formed between the carboxyl groups of galactosyluronic acid residues in one homogalacturonan (HG) chain and the O-2 (and/or O-3) hydroxyl groups of galactosyluronic acid residues in another homogalacturonan chain.

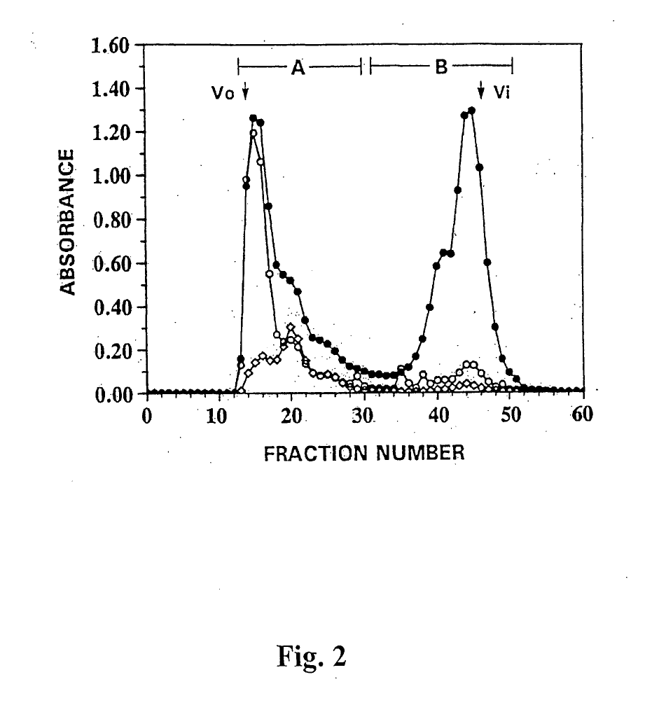

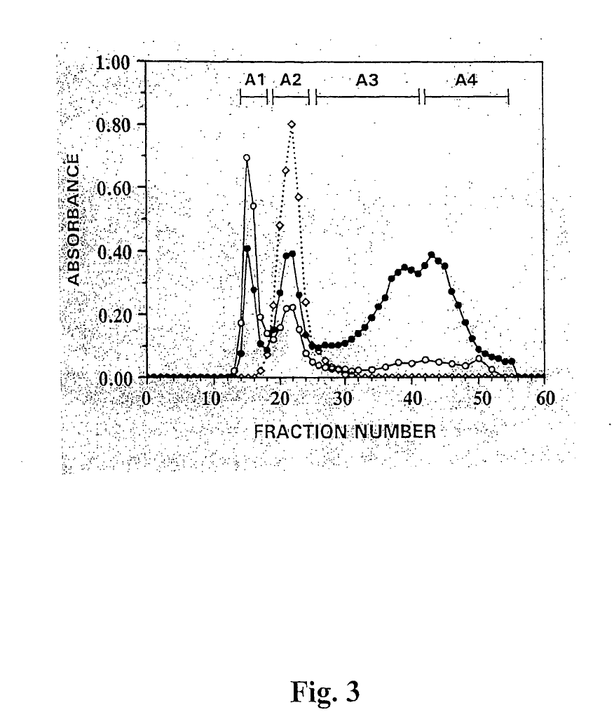

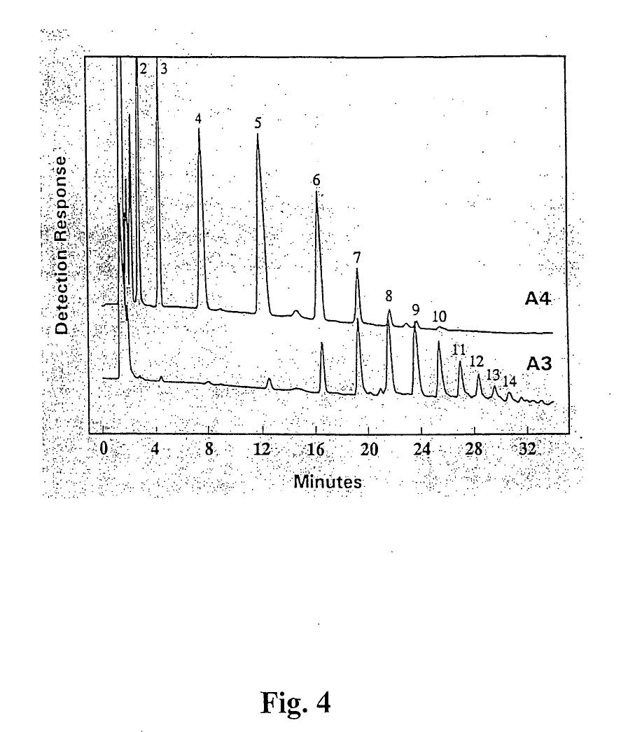

In order to identify an enzyme(s) responsible for hydrolyzing ester bonds (other than methyl esters) that cross-link homogalacturonan chains of pectin, sycamore cells cultured in suspension were initially treated with Asperigillus niger alpha-(1,4)-endopolygalacturonidase (EPG). The EPG-solubilized material was then separated into two fractions on a Bio-Gel P-30 size exclusion column (FIG. 2). The fraction containing the smaller molecular weight (fraction B) consisted of partially methyl-esterified oligogalacturonides. Fraction A was de-esterified by cold-base treatment at 0° C., pH 12 for 4 hours and separated into four fractions by using the same Bio-Gel P-30 column (FIG. 3). Fraction I was shown to be composed of rhamnogalacturonan I, Fraction II, of rhamnogalacturonan II, Fraction III, of oligogalacturonides with degrees of polymerization (DPs) from 6-16, and Fraction IV, of oligogalacturonides with DPs 1-8 (FIG. 4). The oligogalacturonides in fractions III and IV were passed through the P-30 column and eluted at a size equivalent to polygalacturonides (homogalacturonan). The DPs of several rungs of the oligogalacturonide ladder were determined by comparing their migration rates with those of homogeneous oligogalacturonides whose DP was established by mass spectrometry [York et al. (1985) Meth. Enzymol. 118:3-40].

The conditions for treating fraction A with cold base were selected to maximize the hydrolysis of esters and minimize ,β-elimination of the glycosyl anion from C4 of methyl-esterified galactosyluronic acid residues. The absence, following the base treatment, of increased absorbance of ultraviolet light at 235 nm, was taken as evidence that β-elimination did not occur during the cold-base treatment, since β-elimination results in a Δ 4:5 double bond that is conjugated with the carbonyl function at C-6, thereby constituting a chromophore that absorbs ultraviolet light at 235 nm.

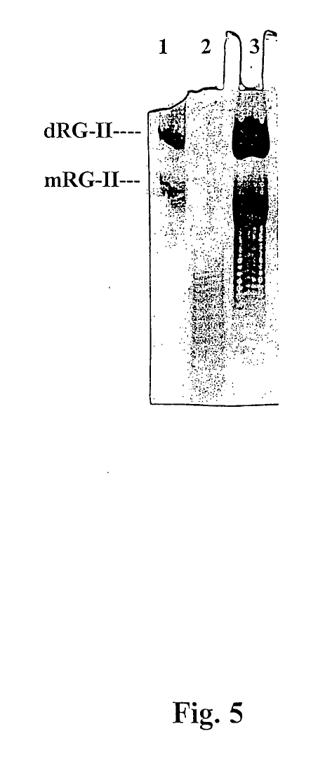

The conclusion that the homogalacturonan chains observed in the gels after cold base treatment were fragmented by hydrolysis rather than by β-elimination was further supported by mass spectral analysis of oligogalacturonides in fractions III and IV. The mass spectral analysis showed that the oligogalacturonides generated by the cold base treatment had molecular weights expected of a hydrolysis reaction, not those of a β-elimination reaction, i.e., β-elimination would result in each oligogalacturonide weighing 18 mass units less than the corresponding hydrolysis product. These results indicate that homogalacturonan fragments are covalently interconnected by ester cross-links that can be cleaved by the cold base treatment, thereby generating the oligogalacturonides in fractions III and IV (see FIG. 4). The oligogalacturonide fragments of fractions III and IV are likely to exist in muro, interconnected by cold-base-labile bonds. The conditions of the cold-treatment of fraction A were similar to those described by Marfa et al. except that the products were analyzed using a polyacrylamide gel electrophoresis (PAGE). As shown in FIG. 5, oligogalacturonides with DPs 10 to 26 and the monomer and dimer forms of RG-II were generated. The ladder of oligogalacturonides disappeared when cold base-treated fraction A was treated with EPG. RG-I, also present in fraction A, was too large to enter the gel.

Lane 1 in FIG. 5 contained the sample from fraction A that had not been treated with cold base. There were no apparent oligogalacturonides in the sample (no ladder), as expected if the oligogalacturonides are cross-linked by esters or are partially methyl-esterified, as this would cause the oligogalacturonides to move more slowly and smear instead of forming bands. However, after cold-base treatment, the oligogalacturonides in fraction A formed a ladder that corresponded to the oligogalacturonides present in fractions III and IV (FIG. 3 and 4) following cold-base treatment of fraction A. (FIG. 5, lane 3).

Fraction A, generated by EPG treatment of cell walls isolated from suspension-cultured sycamore cells, is composed of the three pectic polysaccharides: RG-I, RG-II, and homogalacturonan (HG) or fragments thereof. Although one or more of the components of fraction A appears to be cross-linked via ester bonds, fraction A is too complex to be a useful substrate in our search for an enzyme that hydrolyzes ester cross-links. To find a more amenable substrate, we began investigating commercial pectins as a substrate for our studies.

Commercial pectin is about 90-95% methyl-esterified homogalacturonan. Depending on the particular product, the degree of esterification of the galactosyluronic acid residues of commercial pectin can be as low as 10% or as high as 95%.

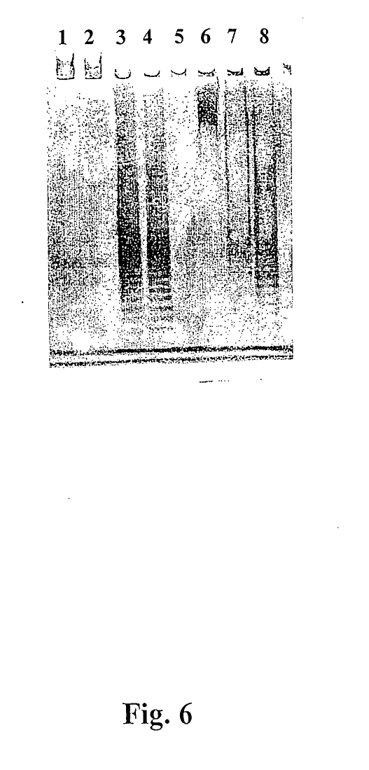

To test whether commercial pectin could function as substrate for an enzyme that hydrolyses ester cross-links, we analyzed untreated samples of Sigma pectin (67% methyl esterified, Sigma Corporation, St Louis, Mo.) and Hercules-pectin (73% methyl esterified, Hercules, Inc., Wilmington, Del.) by PAGE. The untreated pectins did not enter the gel, and no oligogalacturonides were visible (lanes 1 and 5, respectively, of FIG. 6).

Next, a highly purified fungal endopolygalacturonase (EPG) was tested to see whether it could hydrolyze the glycosidic linkage of homogalacturonan of commercial pectins. The results were the same as those with the untreated pectins (lanes 2 and 6 of FIG. 6); no oligogalacturonides were generated. Thus the fungal EPG could not degrade the 73% methyl-esterified commercial pectin. This result is not particularly surprising, as the fungal EPG requires several consecutive unesterified galactosyluronic acid residues before it can cleave the glycosidic linkages of homogalacturonan [Dass et al. (2000) Carbohydrate Res. 326:120-129]. In addition, any oligogalacturonides generated by overnight EPG treatment would be difficult to detect in the polyacrylamide gel because some of the galactosyluronic acid residues would remain methyl esterified causing the oligogalacturonides to smear rather than form distinct bands. Another plausible explanation for the inability of the EPG to cleave the commercial homogalacturonans is that these polysaccharides are so highly cross-linked that the EPG is sterically prevented from reaching susceptible substrate sites.

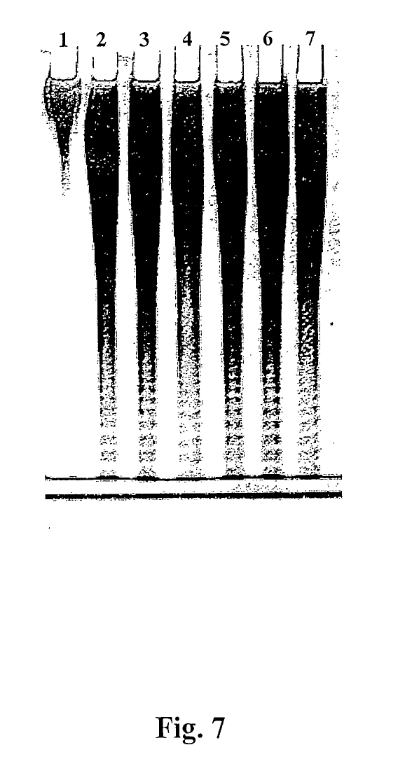

The effect of reaction time of the cold base treatment on the de-esterification of Hercules 73% methyl-esterified pectin was examined next. As shown in FIG. 7, de-esterification was completed in 30 minutes or less. Subsequent experiments have shown, that under the same conditions, de-esterification can be completed in 5 minutes or less.

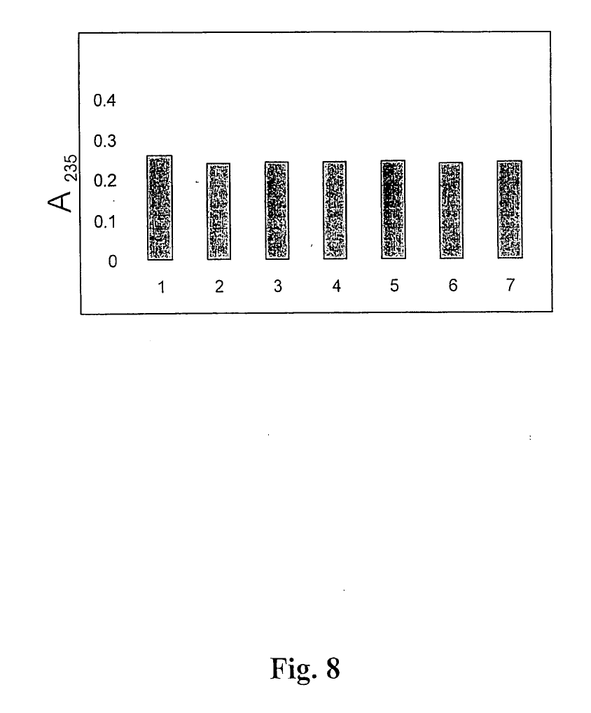

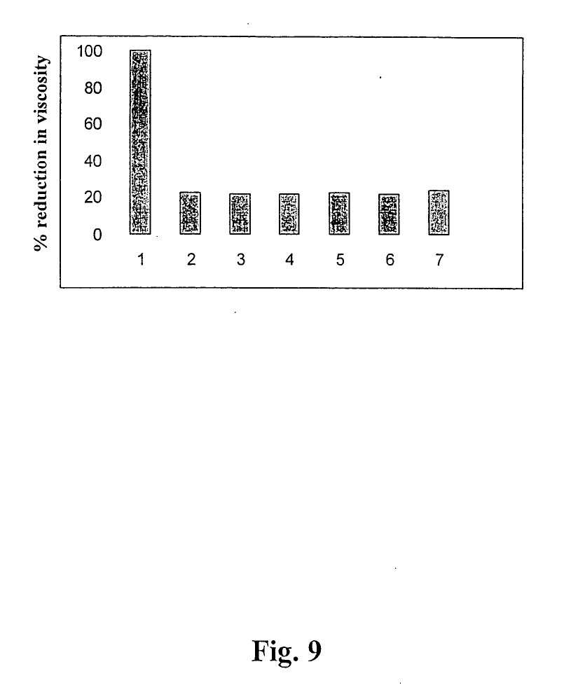

Cold base treatment of pectin at high pH favors de-esterification over β-elimination, while base treatment of pectin at higher temperature and at lower pH (e.g. pH 7) is known to favor β-elimination of glycosyl anions from C-4 of residues activated by methyl-esterified carboxyl groups at C-6. Therefore, base treatments in our experiments were carried out at 0-4° C. and pH 12. Once homogalacturonan is fully de-esterified, samples can be left at pH 12, room temperature for 3 hours without an apparent effect on the products formed at 4° C. (lane 7 of FIG. 7). We examined base-treated samples for β-elimination by measuring their absorbance at 235 nm before and after base treatment (FIG. 8). No change in A₂₃₅ was observed, but if only a small number of β-elimination reactions occurred, it would not have been detected. Base-catalyzed hydrolysis of the methyl esters of galactosyluronic acid residues does not, per se, generate reducing groups but base-catalyzed β-elimination does generate reducing groups. Therefore, we examined the number of reducing groups before and after cold-base treatment of pectin. Cold base treatment of pectin caused no measurable increase in reducing groups. This result, taken together with the absence of an increase at A₂₃₅, confirmed that cold base treatment of homogalacturonan does not generate oligogalacturonides by cleaving glycosidic bonds. We next measured the viscosity of pectin samples prior to and after the cold-base treatment and found that the treatment resulted in 80% loss of the viscosity (FIG. 9). The loss of viscosity concomitant with the formation of a wide range of oligogalacturonides can be best explained by the cleavage of cold base-labile cross-links.

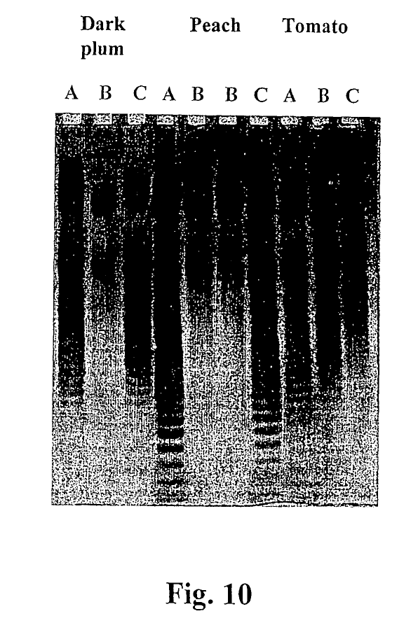

We next set out to identify an enzyme that could release partially methyl-esterified oligogalacturonides from commercial pectin (Hercules 73% methyl-esterified) or cell wall pectin (fraction A of FIG. 2). In order to identify/isolate such an enzyme, several extracts were prepared from the ripe fruit of several plant species and assayed for the hydrolytic activities (FIG. 10), which is termed herein as pectin transesterase (PTE). Oligogalacturonides were generated from fraction A by the extracts prepared from plum, peach, and tomato. Each of the extracts apparently contained sufficient PME to remove methyl esters from the oligogalacturonides so that they formed ladders.

Tomatoes were chosen as a source of enzyme because a great deal of previous research has been carried out on the development and ripening of tomato fruit. Several cell wall-localized tomato fruit enzymes are thought to be involved in development and ripening, including EPG, PME, expansin, and xyloglucan endotransglucanase.

Green tomatoes are known to contain PME but not EPG, while red tomatoes contain both enzymes [Dellapenna et al. (1986) Proc. Natl. Acad. Sci. USA, 83:6420-6424]. It was important to have a supply of PME with no contaminating EPG for our assays of PTE. Thus we prepared extracts of green as well as red tomatoes.

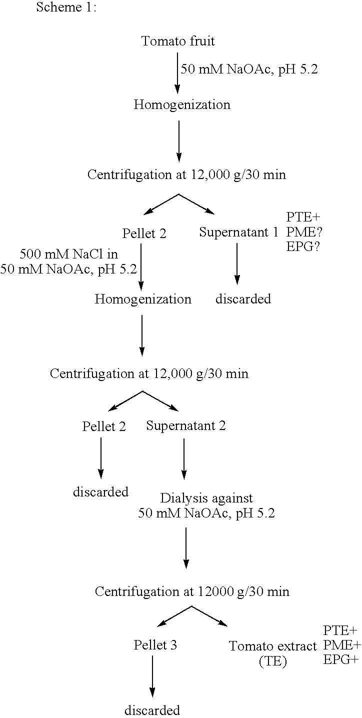

Unripe green and ripe red tomatoes of cultivar UC82B were separately treated as outlined in Scheme 1. Tomato fruit (3 kg) was homogenized at 4° C. in 3 liters of 50 mM sodium acetate, pH 5.2, containing 15 mM β-mercaptoethanol. The homogenizer was a Hamilton Beach 10 Blend Master Mixer operated at high speed with four pulses of 1 minute each with 20 second intervals. The homogenate was centrifuged at 12,000 g for 30 minutes at 4° C. in a Beckman J2-HS centrifuge. Proteins were extracted from the pellet (largely composed of cell walls) by homogenizing the pellet (four pulses for 1 minute with 20 second intervals) at 4° C. in the same buffer containing 500 mM NaCl. The suspension was centrifuged and the resulting supernatant was dialyzed against 50 mM sodium acetate, pH 5.2, at 4° C. The small amount of debris remaining after dialysis was removed by centrifugation as described above and by passage through a Nalgene 0.22 mm filter. The resulting solution is referred to hereafter as “tomato extract” (TE) and more specifically as green tomato extract (GTE) or red tomato extract (RTE).

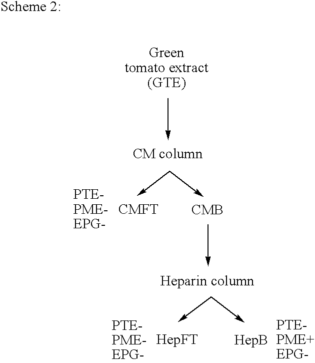

The PME enzyme was partially purified from green tomato extract (GTE) using a Fast Protein Liquid Chromatography (FPLC) system (Pharmacia) according to the steps summarized in Scheme 2. The GTE (100 ml) was loaded via a 50 ml ‘Superloop’, onto a Econo-Pac 5 ml carboxymethyl (CM) ion-exchange column (BioRad Laboratories) that had been equilibrated with 50 mM sodium acetate, pH 5.2. The non-binding material in the GTE was washed through the column with 25 ml of 50 mM sodium acetate, pH 5.2. The material that flowed through the column is referred to as green tomato carboxymethyl flow-through or GT-CMFT. The material bound to the CM column is referred to as the GT-CMB Fraction. The GT-CMB Fraction was eluted from the CM column with 0.5 M NaCl in the sodium acctate buffer.

The GT-CMFT and GT-CMB fractions were collected and assayed for PTE, PME, and EPG activities. PTE and EPG activities were not detected in the GT-CMFT and GT-CMB Fractions. PME activity was detected in the GT-CMB Fraction.

The CMB Fraction, which contained the majority of the PME activity in the GTE, was separated using two 1 ml Pharmacia HiTrap Heparin columns that had been connected in series and equilibrated with 50 mM sodium acetate, pH 5.2. The heparin columns were then washed with 10 ml of the same buffer. Bound proteins were eluted from the CM column with a 50 ml linear gradient of 0-0.35 M NaCl in the sodium acetate buffer and 0.5 ml fractions were collected. The heparin flow-through (GT-HepFT) and bound (GT-HepB) fractions were assayed for PTE, PME, and EPG activities. Fractions containing PME activity were pooled and stored at 4° C.

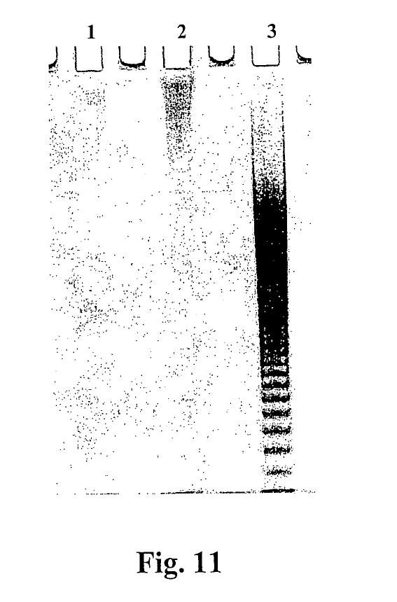

Partially purified green tomato PME did not cleave the ester cross-links of homogalacturonan nor did it cleave the glycosidic bonds of homogalacturonan. The partially purified GT-PME did not cause the release of oligogalacturonides from Hercules pectin (FIG. 11, lane 2). In contrast, when Hercules pectin is treated with a mixture of the GT-PME and RT-PTE (see below), a ladder of oligogalacturonides was generated (FIG. 11, lane 3).

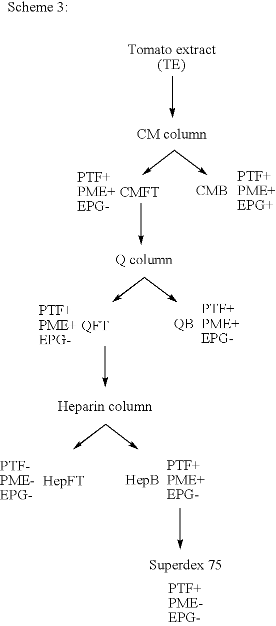

PTE was further purified from RTE as summarized in Scheme 3. Tomato extract (600 ml) was applied via the 50 ml Superloop to the Econo-Pac 5 ml CM column that had been equilibrated with 50 mM sodium acetate, pH 5.2. The material not bound to the column was washed through the column with 50 ml of the sodium acetate buffer. The bound material was eluted from the CM column with 500 mM NaCl in 50 mM sodium acetate, pH 5.2. The flow-through (CMFT) and bound (CMB) Fractions were collected and assayed for PTE, PME, and EPG activities. Both the CMFT and CMB fractions generated oligogalacturonide ladders when analyzed by PAGE, but because the CMFT fraction had nine times less EPG activity than the CMB Fraction, we chose to further purify PTE from the CMFT Fraction.

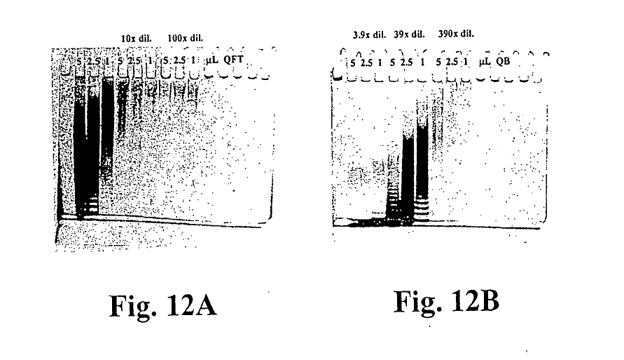

The CMFT Fraction (630 ml) was applied to an anion-exchange HiTrap Q column (1 ml; Pharmacia) that had been equilibrated with 50 mM sodium acetate, pH 5.2. The material that did not bind to the HiTrap Q column was washed through the column with 10 ml of the sodium acetate buffer, and material bound to the column was then eluted with 500 mM NaCl in the sodium acetate buffer. The flow-through (QFT) and bound (QB) fractions were collected and assayed for PTE, PME, and EPG activities. Both fractions contained sufficient PME so that partially methyl-esterified oligogalacturonides generated by PTE and/or EPG would be fully de-esterified and therefore, in the absence of added PME, form ladders of oligogalacturonides (FIGS. 12A and 12B). The QFT and QB fractions had approximately the same amount of PME activity based on the amount of methanol formed when 73% methyl-esterified Hercules pectin was used as substrate.

Neither the QFT nor the QB fraction had detectable EPG activity based on the PAHBAH reducing-group assay [York et al. supra]. The PAHBAH assay was performed using polygalacturonic acid, the substrate most often used for characterizing EPGs. Under the conditions used, EPG activity was detectable if it generated 1 μg of galacturonic acid equivalent reducing groups, an amount that would increase the absorption of ultraviolet light at 414 nm in the PAHBAH assay by 0.1 O.D. This is equivalent to cleaving 2% of the glycosidic bonds in the 50 μg of polygalacturonic acid substrate used in the assay. Smaller amounts of EPG activity can be detected in the more sensitive PAGE assay (see FIG. 12B).

The enzyme activities contained in both the QFT and QB fractions generate oligogalacturonide ladders from Hercules pectin, but the profiles were quite different from each other. The QFT fraction generated the largest amount of oligogalacturonides at the highest concentration assayed (5 μl of the undiluted fraction; FIG. 12B). When the QFT fraction was diluted ten fold, fewer but larger oligogalacturonides were formed. These results are consistent with the expectation that a PTE or EPG was contained in the fraction.

In contrast, the enzymes in the QB Fraction generated the largest amount of oligogalacturonides only after 20- to 50-fold dilution. Further dilution of the QB Fraction resulted in the formation of fewer oligogalacturonides. In contrast, the increased amounts of the QB fraction resulted in the disappearance of the oligogalacturonides. Indeed, the darkly stained area at the bottom of the gel underlying those lanes that contain the largest amounts of the QB fraction supports such activity. This is the result expected of an EPG, but not a PTE. Thus, further efforts to purify a PTE focused on the QFT Fraction.

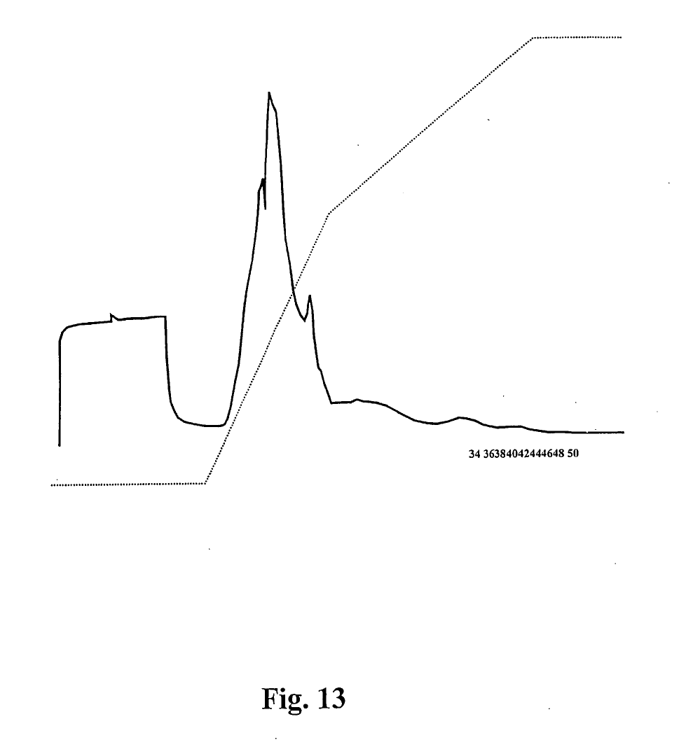

The next step in the purification of the PTE enzyme was to use a HiTrap Heparin column (Pharmacia), which separated the bulk of the remaining protein from the PTE-like activity. The QFT Fraction (100 ml) was applied to two 1 ml heparin columns that were connected in series and had been equilibrated with 50 mM sodium acetate, pH 5.2. The non-binding proteins were washed through the columns with 10 ml of the same buffer. The adsorbed proteins were eluted with a 50 ml linear gradient of 0-0.35 M NaCl in the buffer (FIG. 13). Fractions (4 ml) were collected until the bulk of the protein had eluted from the column, as determined by absorption at 280 nm, at which point the fraction size was reduced to 0.5 ml.

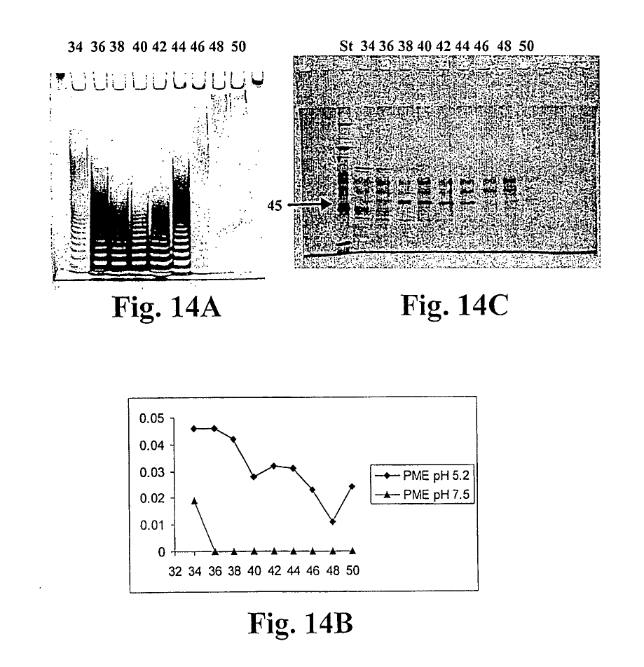

Every second fraction of the heparin column eluate was assayed for the PTE activity (FIG. 14A). Those fractions (36-44) with PTE-like activity were also analyzed for EPG activity with the PAHBAH assay, using polygalacturonic acid as substrate, and for PME activity by measuring the generation of methanol from Hercules pectin. No EPG activity was detected, as was expected since no PAHBAH-detectable EPG activity was seen in the QFT Fraction that was applied to the heparin column. A small amount of PME activity was detected in the PTE-active fractions when the assay was performed at pH 5.2, but not at pH 7.5 (FIG. 14B). Plant PMEs are generally active at pH 7.5 and above so the pectin methyl-esterase associated with the pectin trans-esterase is unusual in this regard.

Every second heparin column fraction from 34 through 50 was analyzed by SDS-PAGE (FIG. 14C). The protein whose presence in the fractions correlated best with the PTE-like activity had a molecular weight of ˜45 kDa (FIG. 14C arrow). Fractions containing PTE-like activity were pooled and reduced to 600 μl using an Amicon Centriprep-10 concentrator (Millipore Corp. Bedford, Mass.).

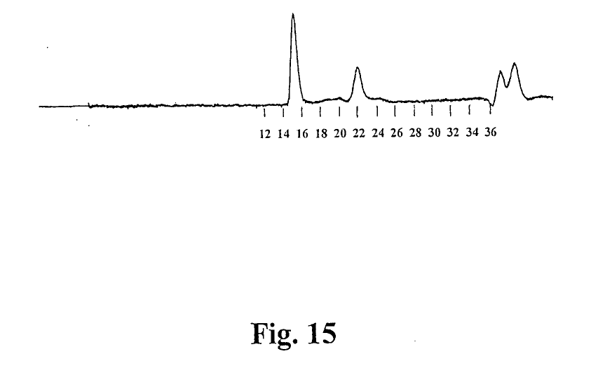

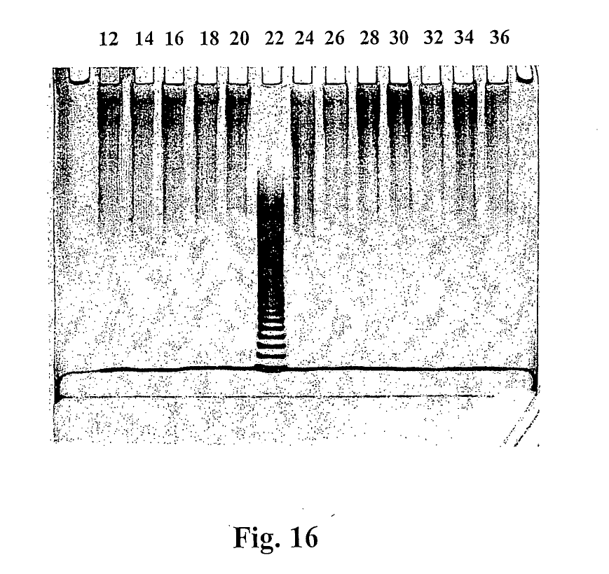

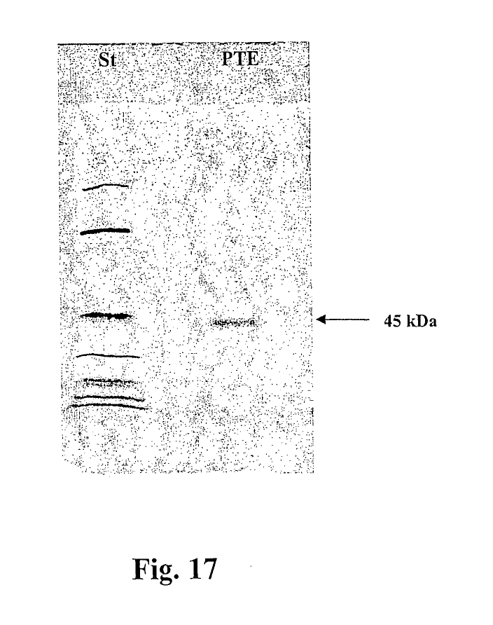

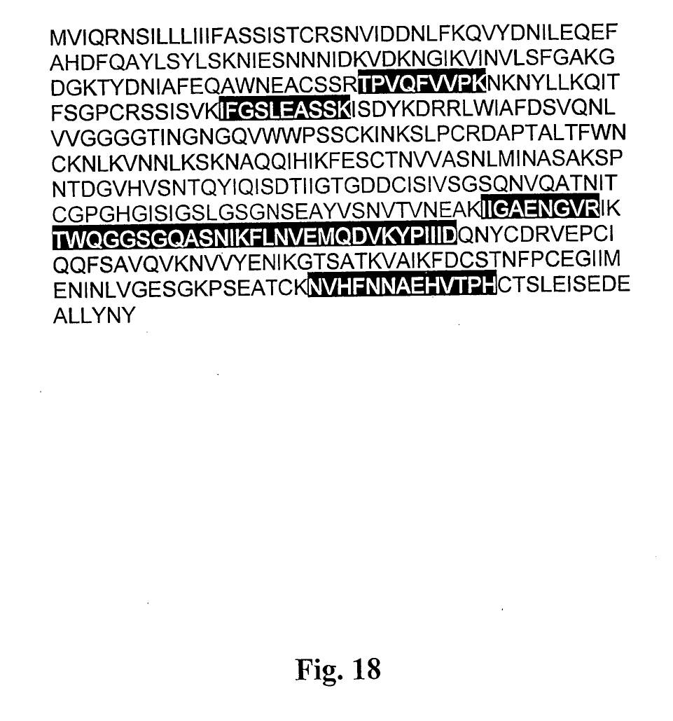

The fraction containing the pectin trans-esterase activity was next subjected to size exclusion chromatography on a Superdex 75 column (1×30 cm; Pharmacia) that had been equilibrated with 0.35 M NaCl in 50 mM sodium acetate, pH 5.2. Two major peaks were separated on this column (FIG. 15). Every second fraction was assayed for pectin trans-esterase activity, and only the protein that eluted in fraction 22 had PTE-like activity (FIG. 16). A portion of fraction 22 was analyzed by SDS-PAGE. One protein band with a molecular weight of ˜45 kDa was detected as shown in FIG. 17. This band was excised and subjected to the amino acid sequence analysis using the standard procedure known in the art. A BlastP protein sequence search of GenBank with the amino acid sequence obtained matched tomato fruit PG2, an unusual α-(1,4)-endopolygalacturonase (FIG. 18).

The studies described so far demonstrate that the newly identified pectin transesterase (PTE) activity is contained in an enzyme, known as endopolygalacturonidase (EPG) up to this point. Both enzymes generate oligogalacturonides, from pectin that form a ladder when subjected to the PAGE analysis, provided the oligosaccharides have been de-esterified by PME. PTE does this without causing a measurable increase in reducing groups as determined by the PAHBAH colorimetric assay. The PAHBAH colorimetric assay is used to determine the number of reducing groups released by the hydrolysis of glycosidic bonds connecting galactosyluronic acid residues.

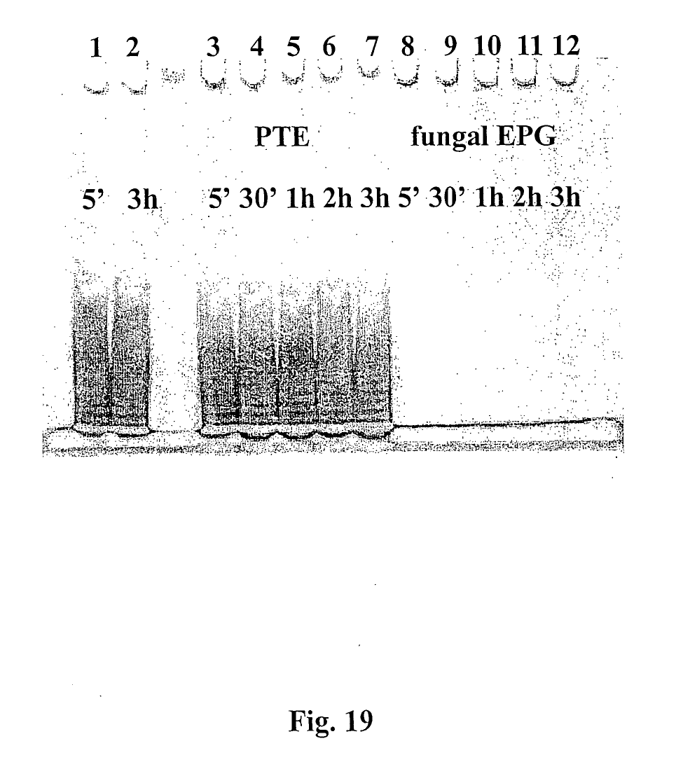

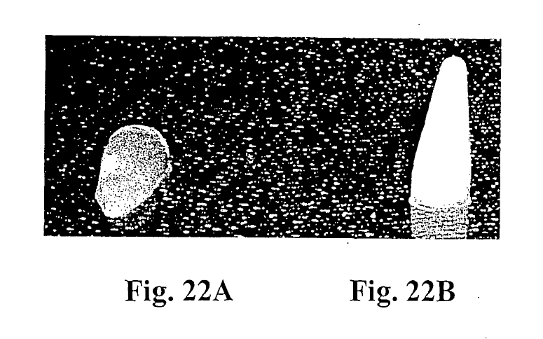

To determine whether the PTE has EPG activity under the same reaction conditions, we used a mixture of oligogalacturonides with degrees of polymerization of 8-20 (the ‘OGmix’) as substrate. We compared PTE's endopolygalacturonase activity to the corresponding activity or a highly purified fungal EPG (Fusarium moniliforme). The results are summarized in FIG. 19. The amount of tomato PTE that is sufficient to generate a clearly visible ladder of oligogalacturonides from pectin in 30 minutes (see FIG. 22) had no apparent ability to reduce the size of the OGmix even after a 3 hour reaction (compare lane 7 with lane 2 in FIG. 19). In contrast, the fungal EPG degrades the OGmix within 5 minutes to completion. Since fungal EPGs are known to have a higher specific activity than plant EPGs, we decided to carry out the similar analysis using the tomato EPG isolated as described herein. To confirm the identity of the purified EPG enzyme, an aliquot (25 μl) of purified tomato EPG was sent for the N-terminal sequence analysis. The amino acid sequence obtained was identical to that of tomato EPG (PG2) published, thus establishing that the purified enzyme is indeed tomato EPG.

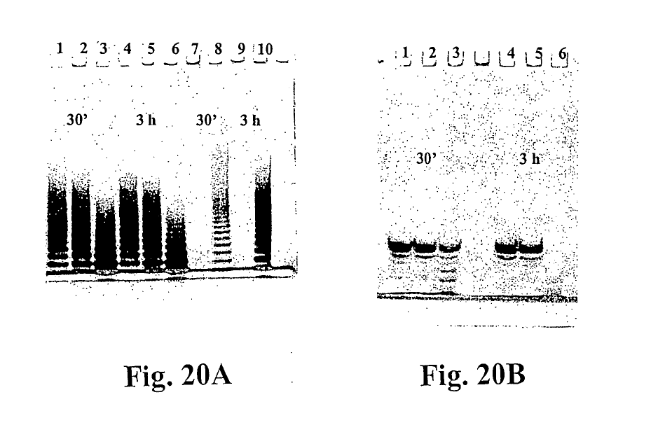

The purified tomato EPG preparation had eight times more protein than the preparation of pectin trans-esterase. Therefore, for the next set of experiments, which were designed to compare the activities of EPG and PTE purified from ripe tomato fruit, we used tomato EPG diluted eight-fold. The amount of enzyme used in these experiments was adjusted so that the PTE would yield a clearly visible ladder of oligogalacturonides from Hercules pectin within 30 minutes (FIG. 20A, lane 8).

Tomato EPG clearly reduced the size of the oligogalacturonides within 30 minutes (FIG. 20A; compare lane 3 with the no enzyme control in lane 1). The PTE has no visible effect on the size of the oligogalacturonides, even after 3 hours (FIG. 20A; compare lane 5 with lane 4).

Hercules pectin is a highly viscous, high molecular weight commercial product. PTE, in the presence of pectin methyl-esterase (PME), reduces the viscosity of Hercules pectin while converting the pectin into a series of oligogalacturonides (FIG. 20A, lanes 8 and 10). PME by itself does not generate oligogalacturonides from pectin (FIG. 11). Although PTE in the presence of PME catalyzes the formation of more oligogalacturonides in 3 hours than it does in 30 min, the PTE shows no ability to reduce the size of the oligogalacturonides (FIG. 20A, compare lane 8 with lane 10).

The abilities of the two enzymes to depolymerize a highly-enriched preparation of tetradecagalactosyluronic acid, a.k.a. the ‘14-mer’ were compared. PTE did not cleave any 14-mer even after 3 hours (FIG. 20B, compare lane 5 with lane 4). In contrast, tomato EPG converted about half the 14-mer to shorter oligogalacturonides in 30 minutes, and converted all the oligogalacturonides to chains of less than 10 galactosyluronic acid residues in 3 hours (FIG. 20B, lanes 3 and 6).

The PTE activity described above is a novel enzyme activity that, in concert with pectin methyl-esterase, converts pectin into a series of oligogalacturonides with degrees of polymerization from 1 to 20 without cleaving glycosidic bonds. In contrast, purified tomato EPG in combination with PME, converts homogalacturonan to mono-, di-, and trigalactosyluronic acids. PTE (with PME) generates the same oligogalacturonide products from pectin as cold base in vitro, and thus PTE and cold base are likely to hydrolyze the same cross-linking esters formed in muro.

PTE and PG2 (i.e., EPG) isolated from ripe tomato catalyze two different reactions; PTE hydrolyzes base-labile putative esters and PG2 hydrolyzes glycosidic bonds. However, as described above, these two enzyme activities are contained in the same protein. Although this finding is unexpected, the fact that plant EPG enzymes often consist of two to four conserved domains with signal peptidase might account for the multiple catalytic activities in a single protein.

Linear, unesterified homogalacturonan, a.k.a. polygalacturonic acid, appears to be the best substrate for EPGs such as PG2. Pectin is a poor substrate for EPGs unless PME is included in the reaction. Pectin is also a poor substrate for PTE unless PME is included in the reaction, but polygalacturonic acid is not a substrate for PTE as polygalacturonic acid does not contain any esters. Polygalacturonic acid, the substrate for EPGs, is the product of the combined catalytic reactions on pectin of PME and PTE. Thus, the product of one enzyme encoded by a PG2 gene is the substrate for the second enzyme encoded by the same gene.

Homogalacturonan is widely believed to be a high molecular weight, partially methylesterified, linear homopolysaccharide. This picture has recently been modified as the result of increased structural knowledge of rhamnogalacturonan II [O'Neill et al. (1996) J. Biol. Chem. 271:22923-22930]. Rhamnogalacturonan II is formed by the attachment of four complex sugar chains to highly conserved positions within seven consecutive galactosyluronic acid residues of the homogalacturonan backbone. The studies described herein indicate that the length of the homogalacturonan chains is highly variable and the degree of cross-links between the HG chains can be regulated by the newly identified PTE activity.

Extraction of pectin-rich plant tissues with mild acid generates polygalacturonic acid, which is composed of various chain lengths (averaging ˜35 residues), as determined by the molar ratio of galactosyluronic acid residues to reducing end groups [Cervone et al. (1989) Plant Physiol. 90:542-548]. This is approximately the same average size chain generated by treating pectin with cold base. PTE, in conjunction with PME, converts pectin into roughly the same length chains, as described herein. Thus the structural picture of pectin that is emerging is one of shorter, variable-length homogalacturonan chains that are cross-linked into high molecular weight networks by carboxylic and borate esters that are more readily hydrolyzed by acid or base than are the glycosidic bonds of galactosyluronic acid residues [Ishii et al. (1996) Carbohydr. Res. 284:1-9]. A question remains as to why the oligogalacturonides generated by PTE are not always degraded by PG2 (EPG) if the two enzyme activities are contained in the same protein.

Tomato EPG is shown to be in at least three glycoprotein isoforms: PG1, PG2 (sometimes referred to as PG2a), and PG2b [Dellapenna et al. (1986) supra; Pressey, R. (1984) Eur. J. Biochem. 144:217-221; Moore et al. (1994) Plant Physiol. 106:1461-1469]. PG1 appears at the onset of ripening. However, in ripe fruit, PG1 accounts for only about 10% of the total EPG [Dellapenna et al. (1986) supra; Smith et al. (1990) Plant Mol. Biol. 14:369-379]. PG2a and PG2b appear 1 to 2 days after PG1, and constitute the majority of EPG in ripe fruit. PG1 has a native molecular weight of 100 kDa, which is much higher than the molecular weights of PG2a and PG2b, which are 45 and 46 kDa [Dellapenna et al. (1986) supra], respectively. Nevertheless, the three EPG isoforms are the products of a single gene [Dellapenna et al. (1990) Plant Physiol. 94:1882-1886].

Pectin methylesterase from ripe tomato fruit is a cell wall-localized enzyme that hydrolyzes methyl esters of galactosyluronic acid residues, releasing methanol and generating free carboxyl groups in the pectic polysaccharide known as homogalacturonan. The studies described below present evidence that the primary function of the tomato fruit enzyme that has been referred to until now as pectin methylesterase (PME) is in reality pectin transester synthase (PTES). The following studies further demonstrate the ability of PME to catalyze the formation of pectin gels and also show that calcium ions are not a critical component of these gels. It is predicted that pectin methyl-esterase forms cross-links between the carboxyl groups of one HG molecule and the C2- or C3-hydroxyls of other HG molecules, and that these cross links are required to form HG gels under physiological conditions.

There are numerous literature reports of the synthesis of esters by esterases. The reversal of the hydrolytic activity has been achieved with great success by lowering the activity of water. In order to test whether a variety of tomato extracts possess PTES activity, the ability to increase the viscosity of the pectin solution was tested. For these experiments, 2-5 milligrams of Hercules pectin (73% methyl-esterified) were dissolved in 800 μl of 0.1 M sodium phosphate, pH 7.3, combined with 200 μl of acetone diluted 1:1 (v/v) with the phosphate buffer. The fractions of tomato extracts tested included a carboxymethyl bound (CMB) fraction (see Scheme 2). The CMB fraction formed a pectin-dependent gel. No gel formed when the CMB fraction was placed in a boiling water bath for 5 minutes before adding, it to the reaction mixture. This was the first indication of the presence of a pectin transester synthase (PTES) function. In order to purify the PTES activity further, a portion of the CMB Fraction was applied to a HiTrap S Column (2ml; Pharmacia) and the eluant from the HiTrap S column was divided into a total of twelve pooled fractions. We separately pooled fractions containing three pectin methylesterases, each of which formed pectin gels in the presence of acetone. Each of these pooled fractions had high PME activity. Another pooled fraction from the HiTrap S column, which contained peak 6, also had high PME activity, but it did not form a pectin gel in the presence of acetone. However, when the pooled fraction containing peak 6 was applied to a Phenyl Superose (Pharmacia) hydrophobic-interaction column, gelling activity coincided with PME activity. This suggested that the HiTrap S fractions that contained peak 6 also contained a factor that interfered with gelling. It was concluded that there is a strong correlation between PME activity and the PTES, which causes pectin to gel in the presence of 10% acetone. The enzyme responsible for pectin gelation was purified by following PME activity as well as the ability to gel a 0.8% Hercules (73% methyl-esterified) pectin solution containing 10% acetone.

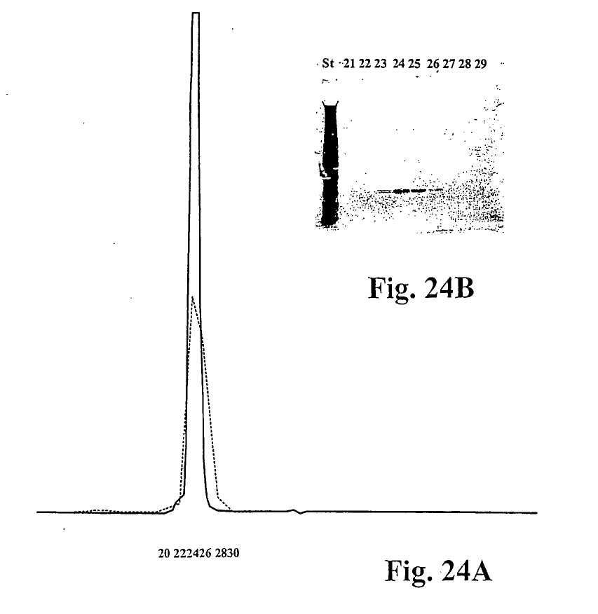

A single protein was purified that had both PME activity and caused pectin solutions to gel. The final step in the purification of the protein was size-exclusion chromatography on a Superdex 75 column (FIG. 24). A portion of the active peak from the Superdex column was subjected to SDS-PAGE. A single, Coomassie blue-stained band was cut from the 12% polyacrylamide SDS gel (FIG. 24B) and sent for amino acid. Twenty peptide sequences, derived from a trypsin digest of the purified protein, were found to match, without error, to sequences within a tomato pectin methylesterase precursor and a pectin methyl esterase (Genbank Accession No. GI 6174913).

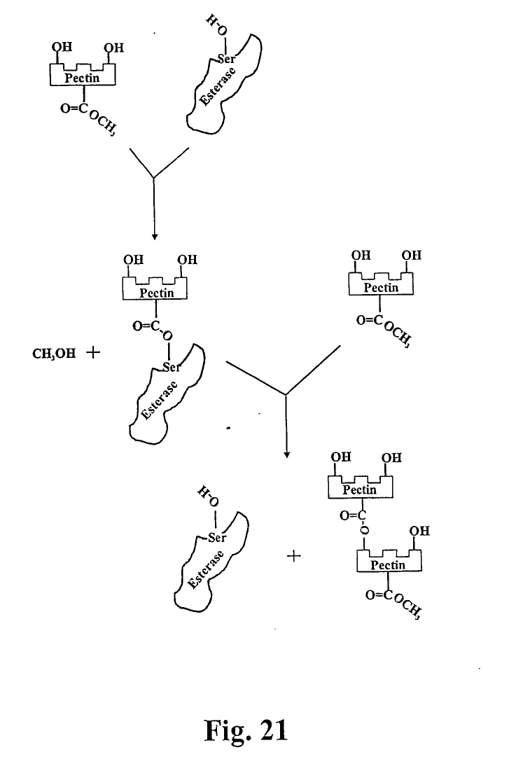

The fact that gelling activity and PME are catalyzed by a single protein indicates that the two activities are carried out by the same enzyme and that the transester synthase is mechanistically the same reaction as that catalyzed by the methyl esterase, except that the synthetic reaction transfers the carbonyl portion of the methyl ester to the hydroxyl of a galactosyluronic acid residue rather than to the hydroxyl of water (FIG. 21). The gelling property of the enzyme also supports the existence of a function for creating ester cross-links between HG chains. The gelling property of the enzyme further supports the existence of ester cross-links between HG chains.

High-methoxy pectins at a concentration of 1.5% and above will, in the absence of enzymes, form gels in the presence of high concentrations of molecules that lower water activity, such as acetone, ethanol, and methanol. When 0.8% pectin and 10% acetone, were used in an assay, a gel formed only in the presence of the enzyme. It showed enzymatically-catalyzed gelation with 0.8% pectin and 10% acetone with pectins that had various degrees of methyl-esterification; namely, 10% methyl-esterified pectin, 30% methyl-esterified pectin, 63-67% methyl-esterified pectin, 73% methyl-esterified pectin and 89% methyl-esterified pectin. All of these pectins gelled in the presence of PME/PTES. None of these pectins gelled when dissolved at a concentration of 0.8% in the absence of the enzyme.

It was discovered that the PME/PTES causes a 2% pectin solution in 0.1 M sodium phosphate, pH 7.3, to gel in the absence of acetone or other molecules that would reduce the activity of water (water activity reducing agents). The reaction was as fast as the reaction observed in the presence of acetone (FIG. 22).

Homogalacturonan, with all its methyl esters removed, is called polygalacturonic acid (PGA). It is predicted that the energy of the methyl esters drives the in muro synthesis of interchain transesterester bonds, and PME/PTES cannot cause polygalacturonic acid to gel. In fact, PME/PTES did not cause a 2% solution of polygalacturonic acid in 0.1 M sodium phosphate, pH 7.3, to gel. The same experiment was tried in the presence of 10% acetone, but the addition of the acetone caused PGA to precipitate. Indeed, acetone is used to precipitate homogalacturonans from solution.

Pectins are known to gel in the presence of relatively high concentration of calcium ions. A well-known egg-box model in which calcium sits between pectin chains forming junction zones has, for more than 25 years, been the accepted “mechanism” for gel formation [Rees, D. A. (1972) supra]. The concentration of calcium ions required to cause low methoxy pectins to gel is on the order of 350 to 500 mM. Four pectin samples, with different degrees of methyl-esterification were analyzed for calcium content. Two samples, 10% methyl-esterified pectin (Sigma), and 30% methyl-esterified pectin (Hercules) contained measurable amounts of calcium. A 2% solution of the 10% methyl-esterified Sigma pectin contains 1.4 mM calcium ions, while a 2% solution of 30% methyl-esterified Sigma pectin contains 1.4 mM calcium ions, while a 2% solution of 30% methyl-esterified Hercules pectin contains 0.7 mM calcium ions. The other pectins analyzed, 73% methyl-esterified Hercules pectin and 89% methyl-esterified Sigma pectin had, at most, trace amounts of calcium. Thus, even the highest concentration of calcium present in the pectin samples is 100- to 1000-fold, too low to cause the pectin samples to gel. These studies indicate that calcium ions are not involved in the PME/PTES-catalyzed gelling of pectin.

Ethylene glycol-bis(β-aminoethyl ether)N,N,N′N′-tetraacetic acid (EGTA) is an excellent chelator of calcium ions. The addition of 100 mM EGTA did not interfere with the formation of pectin gels in the presence of PME/PTES. Furthermore, the addition of 100 mM calcium chloride to 0.8% solutions of low methoxy pectins containing 10% acetone did not result in the formation of a gel unless PME/PTES was also present, in which case the gel formed regardless of whether calcium chloride was added. Finally, the addition of 500 mM calcium chloride to a 2% solution of 10% methyl-esterified pectin (Sigma) or 73% methyl-esterified pectin (Hercules) pectin did not, at pH 7.3, result in the formation of a gel. Gel formation under our reaction conditions requires the addition of PME/PTES.

The purification scheme used to purify PME was initiated by following the extraction protocol and ammonium sulfate fractionation procedure described by Harriman et al. for purifying red tomato PME [Harriman et al. (1991) Plant Physiol. 97:80-87]. Red Premium tomatoes from Publix (4286 g) were homogenized in a Waring blender three times for 30 second each at 4° C. in an equal volume (w/v) of ice-cold ultra-pure water. The homogenized tissue was centrifuged at 10,000 g for 20 minutes. The pellet was suspended in an equal volume (w/v) of ice-cold ultra-pure water, homogenized in the Waring blender three more times for 30 second each at 4° C. and centrifuged again at 10,000 g for 20 minutes. The pellet, enriched in cell walls and depleted in cytoplasmic proteins, was extracted with six volumes (w/v) of ice-cold 1 M sodium chloride in ultra-pure water. The suspension was adjusted to pH 6 with 10 M sodium hydroxide, and allowed to stand for 2 hours at 4° C. The suspension was centrifuged again at 10,000 g for 20 minutes at 4° C. The remaining pellet was discarded.

Ammonium sulfate was slowly added to the vigorously stirred extract of the cell walls of ripe tomato until the solution was 35% saturated. The suspension was stirred overnight at 4° C. and then centrifuged at 10,000 g for 20 minutes at 4° C. The pellet was discarded and the supernatant was, by slow addition of ammonium sulfate and with constant stirring, brought to 85% of saturation. The resulting suspension was left overnight at 4° C. with constant stirring. The resulting precipitate was pelleted by centrifugation at 10,000 g for 20 minutes at 4° C. The pellet (40 grams wet weight) was divided into four Falcon 50 ml tubes. Three tubes, containing 9.35 g. 10.46 g, and 10.57 g, were stored at −80° C. The content of the fourth tube, containing 9.73 g, was dissolved in ice-cold ultra-pure water and dialyzed overnight against 10 mM MES buffer, pH 6.5, containing 0.15 M sodium chloride. The buffer was changed twice. The volume of this solution after dialysis was 72 ml.

The dialyzed protein was divided in half and each half chromatographed on a BioRad Econo-Pac 5 ml CM-column that had been equilibrated with 10 mM Mes buffer, pH 6.5, containing 0.15 M sodium chloride. Unbound proteins were washed through the column with the starting buffer and then the bound proteins were eluted with a 50 ml gradient from 0.15 to 1 M sodium chloride in 10 mM Mes buffer, pH 6.5. One ml fractions were collected.

Although a portion of the PME was bound to the Bio Rad CM-column, the CM flow-through (CMFT) fraction was used to purify PME because we were unable to separate the CMB fraction PME from several proteins.

Proteins in the CMFT fraction (150 ml) were precipitated by bringing the solution to 85% of saturation with ammonium sulfate and allowing the resulting suspension to remain overnight at 4° C. with constant stirring. Precipitated proteins were centrifuged at 10,000 g for 20 minutes. The pellet was dissolved in 20 ml of ice-cold ultra-pure water and dialyzed overnight against 10 mM MES buffer, pH 6.5, with two changes of the buffer.

The dialyzed material was rechromatographed on a BioRad Econo-Pac 5 ml CM-column that had been equilibrated with 10 mM MES buffer, pH 6.5. Bound proteins were eluted with a 50 mL gradient of 0 to 0.15 M sodium chloride in 10 mM MES buffer, pH 6.5; 1 ml fractions were collected. A 1-μl sample of every second column fraction, from 2 through 72, was analyzed for PME activity at pH 7.3. A 4-μl aliquot of every third column fraction, from 3 through 81, was analyzed for protein content by SDS-polyacrylamide gel electrophoresis (SDS-PAGE) that was stained with silver (Nu-PAGE 4-12% gel, (NOVEX San Diego, Calif.). Five microliters of samples of every second column fraction, from 16 through 80, were assayed for its ability to cause 0.8% pectin in 0.1 M sodium phosphate, pH 7.3, to gel in the presence of 10% acetone. Fractions 34-54, 60 and 66 gelled within 8 minutes, while fractions 30, 32, 56, 58, 62, 64, and 68 gelled within 18 minutes. CM-fractions 30 through 56 were pooled and, using an AMICON Centriprep (30 kDa cut-off), concentrated the solution to ˜600 μl and then equilibrated with 50 mM sodium acetate, pH 5.2. Throughout the purification procedure, the ability to gel coincided with PME activity.

Concentrated, desalted, enzymatically-active material from the CM column (˜600 μl) was applied to a cation-exchange HiTrap S column (2 ml; Pharmacia) that had been equilibrated with 50 mM sodium acetate, pH 5.2. Proteins that did not bind to the HiTrap S column were washed through the column with the sodium acetate buffer before eluting bound proteins, first with a 50 ml linear gradient from 0 to 0.25 M sodium chloride in 50 mM sodium acetate, pH 5.2 and then with 15 ml of 0.25 M sodium chloride in 50 mM sodium acetate, pH 5.2. One half ml fractions were collected and 1-μl aliquot of every second fraction, from 2 through 122, was analyzed for PME activity at pH 7.3. A 5-μl aliquot of every third fraction, from 18 through 57, was analyzed for protein content by SDS-PAGE (Nu-PAGE 4-12% gel) that was stained with silver. A 5-μl aliquot of every second fraction, from 10 through 70, was assayed for its ability to cause 0.8% pectin in 0.1 M sodium phosphate, pH 7.3, to gel in the presence of 10% acetone. Fractions 32-60 gelled within 5 minutes, while fractions 26-30 and 62-70 gelled overnight. HiTrap S fractions 35 through 70 were pooled and mixed with an equal volume of 3.4 M ammonium sulfate in 50 mM sodium acetate, pH 5.2.

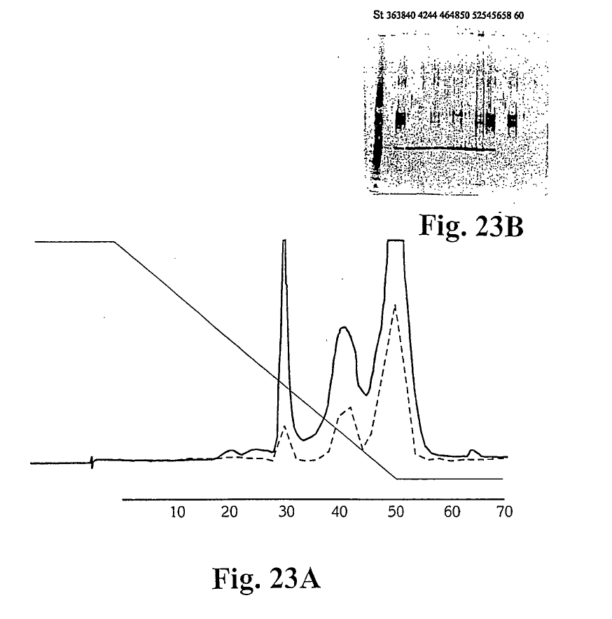

The pooled, ammonium sulfate adjusted, enzymatically-active material, from the HiTrap S column, was applied to a hydrophobic interaction Phenyl-Superose column (Pharmacia) and bound components were eluted with a 25 ml decreasing gradient of 1.7 to 0 M ammonium sulfate in 50 mM sodium acetate, pH 5.2. A 1-μl aliquot from every second 0.5 ml fraction, from 28 through 58, was diluted 50-fold with 0.1 M sodium phosphate, pH 7.3, and a 1-μl aliquot of each diluted sample was analyzed for PME activity at pH 7.3 (FIG. 23A). This is the first step in the purification where the protein content of the eluant is even approximately proportional to the PME activity. The three peaks of PME activity in FIG. 23A may contain PMEs belonging to one or both of the two major groups of PME isozymes found in tomatoes. Tomato PME isozymes reported to be expressed in red tomatoes [Gaffe et al. (1994) Plant Physiol. 105:199-203], falling within the first group have pI values of 8.2, 8.4, and 8.5, while the characteristic of the other group of PME isozymes is pI value of ˜9. A 4-μl aliquot of each 50-fold diluted Phenyl-Superose column fraction, from 36 through 60, was also analyzed for its protein content by SDS-PAGE that was stained with silver (FIG. 23B). A 2.5-μl aliquot of every second Phenyl-Superose column fraction, from 24 through 70, was assayed for its ability to cause 0.8% pectin in 0.1M sodium phosphate, pH 7.3, to gel in the presence of 10% acetone. Fractions 48, 50, and 52 gelled within 3 minutes; fractions 40, 42, and 46 gelled within 5 minutes; fraction 30 gelled within 7 minutes; fractions 38, 44, and 54 gelled within 20 minutes; and fractions 32, 34, 36, 56, and 58 gelled within 2 hours. Phenyl-Superose fractions 47 through 56 were pooled, the pooled fraction was concentrated, and then equilibrated with 75 mM Tris buffer, pH 9.3, using an AMICON Centriprep (30 kDa cut-off, Millipore Corp.).

The concentrated pool of Phenyl-Superose fractions 47-56 (˜600 μl) was applied to a chromatofocusing MonoP column (Pharmacia) that had been equilibrated with the Tris buffer. The unbound proteins were washed through the column with 20 ml of the Tris buffer and then the bound proteins were eluted with 33 ml of Polybuffer 96 (Pharmacia) that had been adjusted to pH 6. A 1-μl aliquot of every second 0.5 ml column fraction, from 2 through 58, was analyzed for PME activity at pH 7.3. A 4-μl aliquot of each MonoP column fraction, from 4 through 10, was analyzed for its protein content by SDS-PAGE that was stained with silver. A single 280 nm absorbing protein peak coincided with the PME and gel-forming activities. A 5-μl aliquot of each MonoP column fraction, from 1 through 15, was assayed for its ability to cause 0.8% pectin in 0.1 M sodium phosphate, pH 7.3, to gel in the presence of 10% acetone. Fractions 6 and 7 gelled within 3 minutes, and fractions 5 and 8 gelled within 20 minutes. MonoP fractions 5-9 were pooled, concentrated using an AMICON Microsep (10 kDa cut-off), and equilibrated with 50 mM Tris, pH 7.5, containing 0.5 M sodium chloride.

The pooled and concentrated MonoP eluant was applied to a Superdex 75 column (Pharmacia). Proteins were eluted from the Superdex 75 column with 50 mM Tris buffer, pH 7.5, containing 0.5 M sodium chloride. A 1-μl aliquot of every second 0.5 mL fraction, from 10 through 32, was analyzed for PME activity at pH 7.3 (FIG. 24A). A 4-μl aliquot of each fraction from 21 through 29 was analyzed for protein content by SDS-PAGE and stained with silver (FIG. 24B). A 5-μl aliquot of each fraction from 20 through 29 was assayed for its ability to cause 0.8% pectin in 0.1 M sodium phosphate, pH 7.3, to gel in the presence of 10% acetone. The aliquot from fraction 24 caused the solution to gel within 3 minutes and the aliquot from fraction 25 within 10 minutes. The single 280 nm absorbing peak that eluted from the Superdex 75 column coincided with the PME and gel-forming activities and with the quantitatively dominant protein detected by SDS-PAGE (FIG. 24B).

The studies described above show that pectin methylesterase can catalyze the formation of a cross-link between homogalacturonan molecules through an ester bond formed between the carboxyl group at C6 of a galactosyluronic acid residue of one homogalacturonan molecule and a hydroxyl group at C2, C3, or C1, of a galactosyluronic acid residue of another homogalacturonan molecule. The result of a sufficient number of such reactions is the formation of a gel (FIG. 22). Thus, treatment of pectin with PME can be used to form pectin gels in the absence of calcium and water activity reducing substance such as acetone.

Ester and amide (peptide) bonds can generally be hydrolyzed by the same enzyme. Thus, most peptidases are esterases. Esterases and peptidases hydrolyze their substrates by transferring the carbonyl function of the ester or amide to a serine or threonine hydroxyl of the enzyme. Hydrolysis is completed by transference of the carbonyl from the enzyme to the hydroxyl of a water molecule. However, many esterases and peptidases favor the transfer of the carbonyl to the hydroxyl of an alcohol or to the amino group of an amine, rather than to water. If this occurs, the esterase or peptidase is acting as a transferase or transester synthase rather than a hydrolase. As disclosed above, tomato pectin methylesterase can transfer the carbonyl function of naturally-occurring methyl esters to the hydroxyl of a galactosyluronic acid residue of another homogalacturonan molecule leading to the formation of a gel of multiply cross-linked homogalacturonan chains.

To determine whether pectin methylesterase can transfer the carbonyl function to amines, commercially available polylysine (Sigma catalog #P-9135) was used as a putative acceptor for the carbonyl function of a methyl-esterified galactosyluronic acid residue. A 4 mg/ml solution of Sigma pectin (10% methylesterified) in 0.1 M sodium phosphate, pH 7.5 and a 4 mg/ml solution of poly-D-lysine in 0.1 M sodium phosphate, pH 7.5, were used. The control sample was prepared by mixing 500 μl of the pectin and 500 μl of the poly-D-lysine solutions and adding 5 μl of 0.1 M sodium phosphate, pH 7.5. A sample testing the ability of the PME to link pectin to poly-D-lysine was the same as the control except that the 5.pl of a solution containing pure tomato PME was added in place of the 5 μl of sodium phosphate buffer. The reaction tubes were ‘vortexed’ to mix the samples and then centrifuted. Within 5 minutes a white precipitate, indicative of the formation of cross-linked material, formed in the tube with the PME. No precipitate formed in the tube without PME.

The experiment was repeated but this time the control sample contained 5 μl of boiled PME (5 minutes at 100° C.) rather than 5 μl of phosphate buffer. As in the previous experiment, a white precipitate, indicative of cross-linking, formed in the sample containing the active PME while the sample containing boiled PME remained clear. Therefore it was concluded that the precipitate formed as the result of the cross-linking enzymatic activity of PME.

The precipitation cannot be explained by the release of methanol and an increase in the number of unsubstituted carboxyl groups, as PME causes a gel to form whether the substrate is 10% methylesterified pectin or 73% methyl-esterified pectin. The transfer reaction and the hydrolysis reaction both release free methanol as a product. Thus far fewer unesterified carboxyl groups are formed by the PME from 73% methyl-esterified pectin than 10% methyl-esterified pectin before it is exposed to PME, yet the 10% methyl-esterified pectin, like the 73% esterified pectin, must be treated with PME in order for it to gel. These data indicate that PME is a transfer synthase that generates a cross-linked gel. It is concluded that the precipitate formed is a polymer of homogalacturonan cross-linked to polylysine via amide linkages.

Next, it was determined that the precipitate formed contained both homogalacturonan and polylysine. This was done by measuring what was left in the supernatant solution following pelleting of the precipitate (by centrifugation at 13,000 rpm for 2.5 minutes in an Eppendorf model 5415D centrifuge). Polylysine was quantified by the BioRad protein assay (BioRad Laboratories), and uronic acid was quantified by the Blumenkrantz and Asboe-Hansen assay (1973) (Analytical BioChem 54:484-489). The amount of poly-D-lysine and uronic acid residues in the control sample (no PME present) was taken to be 100%. An overnight incubation of homogalacturonan and polylysine in the presence of enzymatically active PME resulted in the loss (by precipitation) from the supernatant of 99% (by weight) of uronic acid and 45-50% (by weight) of poly-D-lysine. The same result was obtained using poly-L-lysine in place of poly-D-lysine. Furthermore, no precipitate was observed and no pellet was formed when L-lysine was used instead of polylysine. No change in the uronic acid content of the supernatant was observed in samples containing homogalacturonan in the presence or absence of PME when polylysine was absent in the reaction. There was no gelling in the latter control because the homogalacturonan content was too low (2 mg/ml). The white precipitate formed by PME from homogalacturonan and poly-D-lysine became a brown-colored, translucent, tough plastic-like material when air-dried. It is interesting to note that when a mixture of pectin and poly-D-lysine or simply poly-D-lysine is boiled, a denatured protein-like precipitate of poly-D-lysine is formed. However, the same mixture boiled after incubation with enzymatically active PME shows no sign of denaturation, indicating that pectin is keeping polylysine in solution or protecting it from denaturation.

These results confirm that the PME of ripe tomatoes acts as a “transester synthase” rather than as a methylesterase, although the enzyme may have some level of both activities as the same active site in the enzyme could catalyze both reactions. The transester synthase activity can be used in general to synthesize new composite materials comprising homogalacturonan linked to polymers with unsubstituted amines or alcohols. The homogalacturonan—polylysine composite prepared herein is one example. However, one skilled in the art can appreciate that the methods described herein can be used to link any polymer with unsubstituted amines or alcohols to a pectin to form a novel composite material. Those of ordinary skill in the art will appreciate that such composite materials will have a variety of applications by analogy to known cross-linked polymers.

Chemical methods exist for cross-linking the ester functions of homogalacturonan to alcohols and amines. However, the products of such methods would require extensive characterization, feeding studies, and testing for the presence of the chemical reactants, which would likely make chemical cross-linking commercially unattractive. One can take advantage of nature's ability to cross-link wall polymers. Using enzymes from GRAS organisms to attach the gel-forming poly-anion homogalacturonan to other GRAS organism polymers should lead to a variety of new products that would probably require less rigorous testing for approval by regulatory agencies. For example, pectin could be cross-linked to xyloglucan, which can be isolated in quantity from seeds. Large copolymers of homogalacturonan and xyloglucan should have exceptionally stable gel-forming and extraordinary viscometric properties, due to the propensity of homogalacturonan to form gels and for xyloglucan to form intermolecular hydrogen-bonded complexes. Indeed, xyloglucan can itself form a gel if its terminal galactosyl residues are enzymatically removed. Furthermore, cross-linked chains of homogalacturonan should have better gelling properties than the pectins now commercially available. Homogalacturonan could be attached to cellulose, agarose, or other matrices for new ion-exchange materials. It will also be possible to attach homogalacturonan to galactomannan, starch, polyhydroxybutyrate, or to any number of proteins to form a variety of new materials with novel properties. Moreover, polymers cross-linked by esters are likely to be readily digestible, environmentally friendly, and useful for the slow release of compounds in the body, for example, heparin with anticoagulant or other pharmaceutical properties.

### EXAMPLES

### Example 1

### Plant Material

Tomato (Lycopersicon esculentum Miller, commercial variety UC82B) was grown from seed in Falfard Mix No. 3 with added Cal-Mag Peter's fertilizer. The tomato plants were grown in the green house at ˜21° C., 65% relative humidity with a 12 hour cycle of light and dark.

### Example 2

### Pectin Transesterase (PTE) Assay

Aliquots of fractions to be assayed for PTE activity were added to an Eppendorf tube containing 5 μl of Hercules 73% methylesterified pectin (1 mg/ml in 50 mM sodium acetate, pH 5.2). The volume of the reaction was adjusted to 10 μl by addition of 50 mM sodium acetate, pH 5.2. The PTE reaction was carried out in the presence of GT-PME at room temperature for 3 hours and was terminated by placing the Eppendorf tube in a boiling water bath for 5 minutes. A 2 μl aliquot of a phenol red solution (10 mg of phenol red in 6.67 ml of 1.9 M Tris-HCl, pH 6.8, 10 ml of glycerol, and 3.3 ml of distilled water) was added to each reaction mixture and the mixture was analyzed on a 24% polyacrylamide gel. De-esterified oligogalacturonide bands on a gel were visualized by fixing and staining with Alcian blue, followed by staining with silver. The details of this protocol can be found in Corzo et al. (1991) [Electrophresis 12:439-441].

### Example 3

### Pectin Methyl Esterase (PME) assay

PME assay at pH 7.5

PME activity was measured based on the quantitative analysis of the methanol released by PME. Alcohol oxidase was used to convert the methanol to formaldehyde, which was derivatized and then analyzed colorimetrically [Kalvons et al. (1986) J. Agric. Food Chem. 34:597-599]. Samples to be assayed in ELISA plates for PME activity at pH 7.5 were added to 4 μl of 2% pectin in 0.1 M sodium phosphate, pH 7.5, and 50 μl of diluted alcohol oxidase from Pichia pastoris (1 unit/ml in distilled water; Sigma). The final volume was adjusted to 100 μl and 0.1 M sodium phosphate, pH 7.5, and the reaction incubated at room temperature for 30 minutes. After incubation, 100 μl of acetylacetone solution (0.02 M 2,4-pentanedion in 2 M ammonium acetate and 0.05 M acetic acid) was added to each well.

PME Assay at pH 5.2

Samples to be assayed for PME activity at pH 5.2 were added to an Eppendorf tube with 4 μl of 2% pectin in 0.01 M sodium phosphate, pH 5.2. The Eppendorf tubes were spun for 10 seconds at high speed in a microfuge, and then allowed to react at room temperature of 30 minutes. The reactions were terminated by placing the tubes in a boiling water bath for 5 minutes and then allowed to cool to room temperature. Alcohol oxidase from Pichia pastoris (1 unit/ml in 0.1 M sodium phosphate, pH 7.5) was added to each sample making a final volume of 100 μl. The reaction mixtures were incubated at room temperature for 30 minutes. After incubation, 100 μl of acetylacetone solution (0.02M 2,4-pentanedion in 2 M ammonium acetate and 0.05 M acetic acid) was added to each sample, which was incubated at 60° C. for 15 minutes and then allowed to cool to room temperature. The content of each Eppendorf was transferred to the well of an ELISA plate and the absorption at 414 nm was measured by Titertek Multiscan MCC/340 densitometer (Research Triangle Park, North Carolina).

### Example 4

### Endopolygagacturonase (EPG) Assay

Endopolygalacturonase activity was assayed by the PAHBAH (p-hydroxybenzoic acid hydride) reducing group assay as described in York et al. (1985) [Meth. Enzymol. 118:3-40]. Aliquots (5 μl) of the fractions to be assayed were added to 50 μl of polygalacturonic acid (1 mg/ml; Sigma) in 50 mM sodium acetate, pH5.2. The reaction mixtures were incubated at room temperature for 30 minutes. One hundred fifty μl of freshly prepared PAHBAH solution (5 g of PAHBAH in 100 ml of 1.5% HCl mixed with 0.5 M NaOH in the ratio 1:4) was added to each reaction and the reaction tubes were placed in a boiling water bath for 10 minutes, and then allowed to cool to room temperature. The absorption at 414 nm was determined for each reaction mixture with a Titertek Multiscan MCC/340 densitometer.

### Example 5

### Cold-Base De-Esterification

Cold-base de-esterification was carried out as described in Marfa et al. (1991) [The Plant J. Cell Mol. Biol. 1(2):217-225]. Samples to be de-esterified were dissolved in ultra pure water and brought to 4° C. The solution was adjusted to pH 12 with cold 1 M NaOH. This pH was maintained at 4° C. for 4 hours by addition, as needed, of 0.1 M NaOH. After 4 hours, the pH was adjusted to 5.2 with glacial acidic acid.

### Example 6

### Viscosity Assay

The viscosity of pectin solutions (3 mg/ml in water) was determined at 37° C. with an Anton PAAR KG automated microviscometer (PAAR Physica, Inc., USA, Spring, Tex.) based on the “rolling ball principle.” Samples were placed in the capillary containing a gold covered steel ball. The viscosity was measured as described by the instrument manufacturer at a 45° angle with 5 repetitions each. The results were compared to the viscosity of water, determined under the same conditions.

### Example 7

### Protein Analysis

Proteins were separated on a 4-15% mini gel (Pharmacia) under standard denaturing conditions (SDS-PAGE) using PhastSystem electrophoresis instrument (Pharmacia). Molecular weight standards were purchased from BioRad Laboratories.

### Example 9

### Peptide Sequencing

A purified PTE protein band, visible in a 12% SDS-PAGE gel after Coomassie blue staining, was excised from the gel, destained with 10% acetic acid in methanol, and sent to the Harvard Microchemistry Laboratory, Cambridge, Massachusetts, for trypsin-based peptide sequencing. An aliquot (25 μL) of purified tomato EPG was sent to University of Michigan analytical facility for sequencing.

While the foregoing specification teaches the principles of the present invention, with examples provided for the purpose of illustration. It will be understood that the practice of the invention encompasses all of the usual variations, adaptations or modifications, as come within the scope of the following claims and its equivalents.

All references cited in the present application are incorporated in their entirety herein by reference to the extent not inconsistent herewith.

1. A method for forming an ester or amide bond between a monomeric or polymeric ester or acid or salt thereof and a monomeric or polymeric alcohol or amine which comprises the step of treating the ester, acid or salt thereof with a plant pectin transester synthase in the presence of the alcohol or amine under conditions suitable to form the ester or amide bond.

2. The method of claim 1 wherein the ester, acid or salt thereof is a polymer.

3. The method of claim 2 wherein the polymer is a homogalacturonan.

4. A method for preparing a polymer comprising treating a homogalactuonan and a polymer comprising one or more ester or amine groups with a pectin transester synthase under conditions suitable to form at least one covalent linkage between the homogalacturonan and the polymer.

5. A method for preparing a pectin-based mixed polymer comprising treating a homogalacturonan and a polymer comprising one or more ester or amine groups with a pectin transester synthase in the absence of calcium under conditions suitable to form a covalent linkage between the homogalacturonan and the polymer.

6. The method of claims 4 or 5 wherein said polymer has an unsubstituted amine or alcohol group.

7. The method of claims 4 or 5 wherein said polymer is xyloglucan.

8. The method of claims 4 or 5 wherein said polymer is D- or L-polylysine.

9. The method of claims 4 or 5 wherein said pectin transester synthase is isolated from a plant selected from the group consisting of tomato, tobacco and spinach.

10. A polymer composition made according to claims 1-5.

11. A method for preparing a pectic gel comprising treating homogalacturonan or a mix of homogalacturonan with other polysaccharides with a pectin transester synthase in the absence of calcium under conditions suitable to form an intermolecular covalent linkage between homogalacturonons or between homogalacturonan and other polysacchrides.

12. The method of claim 11 wherein said pectin transester synthase is isolated from a plant selected from the group consisting of tomato, tobacco, and spinach.

13. A pectic gel made according to claim 11.

14. A method of regulating the viscosity of a pectin-containing composition comprising treating said composition with a pectin transesterase (PTE) under conditions suitable to hydrolyze an ester bond of homogalacturonan contained in the composition.

15. The method of claim 14 wherein said pectin containing composition is a beverage.

16. The method of claim 14 wherein said PTE is isolated from a plant.

17. The method of claim 16 wherein said plant is selected from a group consisting of plum, peach, and tomato.

18. A method of thinning a pectin-containing beverage comprising treating the beverage with a pectin transesterase (PTE) under conditions suitable to hydrolyze ester bonds of homogalacturonan of the pectin in the beverage.

19. The method of claim 18 wherein said beverage is a fruit or vegetable juice.

20. The method of claim 18 wherein said PTE is isolated from a plant.

21. The method of claim 20 wherein said plant is selected from a plant consisting of plum, peach, and tomato.


SEQUENCE LISTING (1 sequence)

SEQ ID NO: 1 - protein, 456 aa, Tomato
MVIQRNSILLLIIIFASSISTCRSNVIDDNLFKQVYDNILEQEFAHDFQAYLSYLSKNIESNNNIDKVDKNGIKVINVLSFGAKGDGKTYDNIAFEQAWNEACSSRTPVQFVVPKNKNYLLKQITFSVDKNSSISVKIFGSLEASSKISDYKDRRLWIAFDSVQNLVVGGGGTINGNGQVWWPSSCKINKSLPCRDAPTALTFWNCKNLKVNNLKSKNAQQIHIKFESCTNVVASNLMINASAKSPNTDGVHVSNTQYIQISDTIIGTGDDCISIVSGSQNVQATNITCGPGHGISIGSLGSGNSEAYVSNVTVNEAKIIGAENGVRIKTWQGGSGQASNIKFLNVEMQDVKYPIIIDQNYCDRVEPCIQQFSAVQVKNVVYENIKGTSATKVAIKFDCSTNFPCEGIIMENINLVGESGKPSEATCKNVHFNNAEHVTPHCTSLEISEDEALLNY